(19) United States
(12) Patent Application Publication
(10) Pub. No.: US 2012/0028837 A1
(43) Pub. Date: Feb. 2, 2012

(54) RAPID SCREEN FOR REPRODUCTIVE TOXICANTS

(75) Inventors: Monica Colaiacovo, Newton, MA (US); Patrick Allard, Cambridge, MA (US)

(73) Assignee: PRESIDENT AND FELLOWS OF HARVARD COLLEGE, Cambridge, MA (US)

(21) Appl. No.: 13/018,681

(22) Filed: Feb. 1, 2011

Related U.S. Application Data
(60) Provisional application No. 61/311,635, filed on Mar. 8, 2010.

(51) Int. Cl. C12Q 1/18; C40B 30/06; G01N 33/53; C12Q 1/66

(52) U.S. Cl. 506/10; 435/32; 435/8; 435/7.1

(57) ABSTRACT

Disclosed herein are methods to assess the biological safety of an agent. The method involves contacting one or more test agents to a culture of C. elegans and analyzing the culture for meiotic disruption, wherein an increase in meiotic disruption of the C. elegans, indicates that the test agent(s) has reduced biological safety to mammals. An increase in meiotic disruption of the C. elegans also indicates a likelihood that the test agent is a reproductive toxicant in higher animals, such as humans. Also disclosed are methods to identifying disruptors of fat homeostasis in a mammal. The method involves contacting one or more test agents to a culture of C. elegans and analyzing the culture for fat content, wherein a change in the fat content, as compared to an appropriate control, indicates that the test agent(s) is a likely disruptor of mammalian fat homeostasis.

### RELATED APPLICATIONS

This application claims the benefit under 35 U.S.C. §119(e) of U.S. Provisional patent application Ser. No. 61/311,635, filed Mar. 8, 2010, the contents of which are herein incorporated by reference in their entirety.

### GOVERNMENTAL SUPPORT

This invention was made with government support under GM072551 awarded by the National Institutes of Health. The government has certain rights in this invention.

### FIELD OF THE INVENTION

The present invention relates to the field of toxicology and the screening of compounds to identify substances which are toxic or have undesired biological effects.

### BACKGROUND OF THE INVENTION

While it was long thought that the human embryo was sheltered from exogenous chemical harm, the tragedies of thalidomide and the synthetic estrogen diethylstilbestrol (DES) have since clearly highlighted the sensitivity of the embryo and developmental processes to the environment. Meiosis, in particular, is exceptionally sensitive to environmental factors. For example, in yeast, nutritional stress is known to modulate meiotic recombination rates (Abdullah et al., Proc Natl Acad Sci USA. 2001; 98(25):14524-9. PMCID: 64715.). In addition, in a variety of species, temperature fluctuations can dramatically alter proper chromosome segregation during meiosis (Chan et al., PLoS One. 2009; 4(10):e7284. PMCID: 2757900; Grell R F. Environ Health Perspect. 1979; 31:33-9. PMCID: 1637661; Rose, Genetics. 1979; 92(2):409-18. PMCID: 1213967). Besides variation in natural environmental conditions, more artificial conditions such as exposure to DNA damaging chemicals and irradiation have been shown to alter meiosis in many species including mammals (Hanneman et al., Proc Natl Acad Sci USA. 1997; 94(16):8681-5. PMCID: 23075; Poorman-Allen et al., Mutagenesis. 1990; 5(6):573-81; Takanami et al., Nucleic Acids Res. 2000; 28(21):4232-6. PMCID: 113154). Our knowledge of environmental effect on human meiosis is more limited, but we know that recombination rates can show extreme variation depending on the population being tested (Kong et al., Science. 2008; 319(5868):1398-401; McVean et al., Science. 2004; 304(5670):581-4) and that the rate of chromosome non-disjunction is correlated with environmental factors such as social-economical status, smoking and intake of contraceptive drugs (Hunt P A. Biochem Soc Trans. 2006; 34(Pt 4):574-7; Yang et al., Genet Med. 1999; 1(3):80-8). Predictably, meiotic abnormalities are very common in humans. For example, aneuploidy, or presence of an abnormal number of chromosomes, can be detected in 20% of all oocytes, 35% of clinically recognized miscarriages and 4% of stillbirths (Hunt P A, Hassold T J. Trends Genet. 2008; 24(2):86-93).

Despite the severe outcome of impaired meiosis, the screening and analysis of environmental toxicants for their ability to disrupt this process have been particularly challenging, mainly because meiosis is a complex cellular program that cannot be recapitulated in a tissue culture setting. The study of mammalian female meiosis is especially arduous as it spans from several months in mice to several decades in humans. It is initiated early on during embryogenesis, where key events guiding chromosomal segregation and exchange take place, and is only completed at puberty during ovulation. Each of these crucial steps of meiotic prophase I, namely chromosome pairing, synapsis and recombination, occur at specific stages of development within the confines of the embryonic gonad and are therefore not easily accessible for study. Due to time, cost, experimental and ethical constraints, the study of environmental effect on the process of meiosis in mammals is therefore highly challenging. The tools to efficiently and comprehensively interrogate the chemical environment for its effect on mammalian reproduction and meiosis are not currently available. A fast and reliable multicellular meiotic toxicological model relevant for mammalian meiosis does not currently exist.

### SUMMARY OF THE INVENTION

One aspect of the present invention relates to a method for assessing the biological safety of one or more test agents. The method comprises contacting the one or more test agents to a culture of C. elegans and analyzing the culture for meiotic disruption. An increase in meiotic disruption of the C. elegans, indicates that the test agent(s) has reduced biological safety to mammals. In one embodiment, assessing the biological safety of one or more test agents comprises providing one or more test agents, and a culture of C. elegans, contacting the one or more test agents to the C. elegans within the culture, and analyzing meiosis in the C. elegans and comparison to an appropriate control to thereby determine the presence or absence of meiotic disruption. An increase in meiotic disruption of the C. elegans, compared to the control, indicates the test agent has reduced biological safety to mammals.

Another aspect of the present invention relates to a method to identify a likely mammalian reproductive toxicant. The method comprises contacting the one or more test agents to a culture of C. elegans and analyzing the culture for meiotic disruption, wherein an increase in meiotic disruption of the C. elegans, indicates the test agent(s) is a likely mammalian reproductive toxicant. In one embodiment, identifying a likely mammalian reproductive toxicant comprises providing one or more test agents, and a culture of C. elegans, contacting the one or more test agents to the C. elegans within the culture, and analyzing meiosis in the C. elegans and comparison to an appropriate control to thereby determine the presence or absence of meiotic disruption. An increase in meiotic disruption of the C. elegans, compared to the control, indicates the test agent is a likely mammalian reproductive toxicant.

In one embodiment of the herein described methods, analyzing meiosis is by assessing the culture for % male embryos in adult C. elegans midventral sections, and meiotic disruption is indicated by an increase in % male embryos.

In one embodiment of the herein described methods, analyzing meiosis is by assessing the culture for number of apoptotic nuclei, for RAD-51 foci, or levels of ATL-1, in adult germlines (e.g., zones 6 and 7, which correspond to late pachytene stages of meiosis), and meiotic disruption is indicated by an increase in RAD-51 foci, an increase in the number of apoptotic nuclei or an increase in the level of ATL-1.

In one embodiment of the herein described methods, analyzing meiosis is by analyzing the culture for chromosome morphology during diakinesis, and meiotic disruption is indicated by aberrant chromosome morphology during diakinesis.

In one embodiment of the herein described methods, analyzing meiosis is by analyzing the culture for disassembly of the synaptonemal complex from late pachytene through diakinesis, and meiotic disruption is indicated by aberrant disassembly of the synaptonemal complex at any stage from late pachytene through diakinesis.

Another aspect of the present invention relates to a method for assessing one or more test agents for the ability to disrupt meiosis in C. elegans. The method comprises providing one or more test agents, and a culture of C. elegans, contacting the one or more test agents to the C. elegans within the culture, and determining the percentage of male embryos in adult mid-ventral sections. An increase in the percentage of male embryos, compared to an appropriate control, indicates the agent disrupts meiosis in C. elegans.

In one embodiment of the herein described methods, the C. elegans contains a male specific nucleic acid reporter construct in expressible form. In one embodiment, the nucleic acid reporter construct comprises a promoter from a male specific gene, operably linked to a nucleic acid encoding a reporter protein. In one embodiment, analyzing for meiotic disruption comprises detecting an increase in the reporter protein as compared to an appropriate control. In one embodiment, the gene is xol-1. In one embodiment, the reporter is selected from the group consisting of GFP and luciferase. In one embodiment, the male specific nucleic acid reporter construct is xol-1-GFP. In one embodiment, the C. elegans further contains a ubiquitously expressed reporter construct.

In one embodiment of the herein described methods, the culture of C. elegans is grown in a liquid culture medium designed for high-throughput screening. In one embodiment, the C. elegans culture comprises about 25 worms/well, in one or more wells of a 96-well plate.

In one embodiment of the herein described methods, the agent(s) is contacted to the culture by incorporation into the growth medium, at a concentration of from about 1 μM to about 100 mM.

In one embodiment of the herein described methods, the agent(s) is contacted to the C. elegans for a duration of about 1-6 days. In one embodiment the agent(s) is contacted to the C. elegans for a duration of about 4-6 days. In one embodiment the agent(s) is contacted to the C. elegans for a duration of about 4 days. In one embodiment the agent(s) is first contacted to the C. elegans at the developmental stage ranging from eggs to 17 days post L-4.

In one embodiment of the herein described methods, the agent is first contacted to the C. elegans at a developmental stage from eggs to 2 days post-L4. In one embodiment the agent(s) is first contacted to the C. elegans as an egg. In one embodiment, the agent(s) is first contacted to the C. elegans at 2 days post-L4.

In one embodiment of the herein described methods, the agent(s) is dissolved in a solvent prior to addition to the C. elegans medium.

In one embodiment of the herein described methods the C. elegans of the culture has a mutation that affects the integrity of its cuticle. In one embodiment, the cuticle mutation is selected from the group consisting of bis-1(nx3), dpy-2(e8), dpy-7(e88) and dpy-10(e128).

In one embodiment of the herein described methods a plurality of agents are co-assessed in a high-throughput assay. In one embodiment, the assessment is made using flow cytometry.

### Definitions

As used herein, operative linkage or operative association, or grammatical variations thereof, of heterologous nucleic acid (DNA/RNA) to regulatory and effector sequences of nucleotides, such as promoters, enhancers, transcriptional and translational stop sites, and other signal sequences refers to the relationship between such DNA and such sequences of nucleotides. For example, operative linkage of heterologous DNA to a promoter refers to the physical relationship between the DNA and the promoter such that the transcription of such DNA is initiated from the promoter by an RNA polymerase that specifically recognizes, binds to and transcribes the DNA. The term “operatively linked” includes having an appropriate start signal (e.g., ATG) in front of the polynucleotide sequence to be expressed, and maintaining the correct reading frame to permit expression of the polynucleotide sequence under the control of the expression control sequence, and production of the desired polypeptide encoded by the polynucleotide sequence.

As used herein, “contacting” refers to bringing an agent of interest into sufficient proximity with an organism to promote uptake of the agent by the organism. Uptake can be, for example, through the cuticle, by ingestion (oral), or by any other means of entry into the body from the surrounding media.

As used herein, it is understood that the term “test agent” can include a plurality of such agents, and will be interpreted as such, based on the context in which it is used.

As used herein, the term “culture of C. elegans” refers to a plurality of the organism grown together. In one embodiment, the culture is synchronized so that the culture contains a homogenous mixture of C. elegans with respect to stage of development and/or age. Generally the culture is uniform with respect to genetic background, including any transgenic modifications contained therein.

As used herein, the term “high-throughput” refers broadly to investigations with a large number of assays such that formatting of each individual sample, minimizing preparation steps and complications, and measuring of the assay results either in parallel or in rapid succession become important. High-throughput tests do not include manual, one-at-a-time assays, such as assays by a single individual in which the preparation, execution, measurement, and data collection for one assay are all completed before the assay on the next agent is done. High-throughput is meant to include, for example, any assays in which a batch of samples (e.g., 24, 96, or 384 test culture samples) are prepared and measured, since formatting the tests in such test culture samples is meant to accelerate the assay process by enabling measurement in parallel or in rapid succession, such as with the assistance of automation.

As the term is used herein, a “solvent” is a solution that is used to dissolve a test agent in an aqueous solution for administration to the C. elegans (e.g., by mixture with the culture media). Solvents, for example, can be non-polar, polar-aprotic, and polar-protic. Non-polar solvents include, without limitation, pentane, cyclopentane, hexane, cyclohexane, benzene, toluene, 1,4-dioxane, chloroform, diethylether. Polar aprotic solvents include, without limitation, dichloromethane (DCM), tetrahydrofuran (THF), ethylacetate, acetone, dimethyl formamide (DMF), acetonitrile (MeCN), dimethylsulfoxide (DMSO). Polar protic solvents include, without limitation, formic acid, N-butanol, isopropanol, N-propanol, ethanol, methanol, and acetic acid. Other useful solvents are N-methyl-2-pyrrolidinone (NMP), benzonitrile, ethylene glycol, ethylene dichloride, pyridine, methyl isobutyl ketone, isooctane, carbon disulfide, carbontetrachloride, and o-xylene. A combination of solvents can also be used.

### BRIEF DESCRIPTION OF THE DRAWINGS

FIG. 1A and FIG. 1B, contains bar graphs. These demonstrate reductions in both the brood size (# of eggs laid) and number of worms that reach a larval stage of development following exposure to the indicated doses of Bisphenol A (BPA) compared to control (Eth.=Ethanol).

FIG. 2 contains two bar graphs. The graph on the left depicts the decrease in mitotic nuclei observed in BPA-exposed gonads compared to control (Eth.=Ethanol). The graph on the right depicts the decrease in phosphorylated histone H3 (p-H3) detected in the mitotic zone of the gonad of BPA-exposed worms compared to control.

FIG. 3A is a drawing that represents the various zones within the C. elegans germline, above two bar graphs. The left bar graphs depict the altered levels of RAD-51 foci detected only in BPA-exposed gonads compared to control (Ethanol). The right bar graph depicts the activation of a CEP-1/p53-dependent DNA damage checkpoint in the germline of BPA-exposed germlines. Numbers on the y-axis represent the mean number of germ cell corpses (apoptotic bodies) detected in the gonad arms scored.

FIG. 4 is a schematic representation of the C. elegans gonad indicating progression from mitosis into meiosis from left to right, and a chart containing data. The chart indicates the percentage of oocytes where chromosome condensation defects were detected following exposure to either BPA or Ethanol (Control).

FIG. 5A is a bar graph. FIG. 5B is a bar graph. FIG. 5C is a bar graph. FIG. 5D is a chart containing data. FIG. 5A compares the # of mitotic nuclei detected in the gonads of Ethanol (Control), the estrogen estradiol (E2) or BPA-exposed gonads. FIG. 5B compares the same exposures described in FIG. 5A except # of phosphorylated histone H3 (pH3) nuclei are scored. FIG. 5C extends the analysis of the number of mitotic nuclei to encompass gonads exposed to a combination of BPA and E2 (at two different concentrations) and BPA and 4-HT (the estrogen receptor modulator 4-hydroxytamoxifen). FIG. 5D demonstrates the frequency of condensation defects observed for chromosomes in late diakinesis oocytes, for the indicated exposures. Taken together, these analyses demonstrate that similar to what is proposed in mammals, BPA also acts as an anti-estrogen in the C. elegans gonad.

FIG. 6 is a bar graph depicting the effect of BPA on the size of the mitotic pool of germ cells. BPA exposure leads to a reduction in the number of mitotic germ cells.

FIG. 7 is a set of bar graphs, showing the inducibility of the luciferase construct in animals exposed to 100 μm and 1 μm nocadozole. Data are represented as fold change over control.

FIG. 8 is three sets of pairs of bar graphs of data showing the sensitivity of xol-1::GFP; nx-3 C. elegans to bisphenol at the indicated concentrations. Data are presented as the percent lethality after 4 days on 1 mM BPA. In the pairs of graphs, the left graphs represented xol-1::GFP, and the right graph represents xol-1::GFP; nx-3. (n=25)

FIG. 9 is a graphical representation of data from experiments devised to test the effect of the indicated agents on the generation of aneuploidies. Results were obtained after 24 hours. The agents are chemotherapeutic agents obtained from NCI.

FIG. 10 is a graphical representation of data from the same experiments as described in FIG. 9, testing the effect of the indicated agents on the generation of aneuploidies. Results were obtained after 4 days.

FIG. 11 shows data representative of results of experiments for identification of male embryos in worm populations with the COPAS Biosort and Profiler (automated, FACS sorting of GFP embryos). The background fluorescence threshold (black bar) is established using a non-fluorescent worm population. Using these parameters, the him-8; xol-1::GFP strain clearly shows an embryonic population expressing high levels of GFP above threshold (arrow).

### DETAILED DESCRIPTION OF THE INVENTION

Aspects of the present invention relate to the finding that the process of meiosis in C. elegans is disrupted as a result of exposure to reproductive toxicants. The finding that reproductive toxicants known to affect mammalian systems disrupt meiosis in C. elegans indicates that the C. elegans can be used as a system to screen for molecules (e.g., pollutants) for such activity, to thereby identify other reproductive toxicants. Importantly, this system can detect a toxic activity at concentrations that are physiologically relevant to mammalian systems. As a specific example, the biological effect of exposure to bisphenol A (BPA), a compound commonly used for the production of polycarbonate and epoxy resins, was characterized in C. elegans. Exposure of C. elegans to BPA disrupted meiosis, as determined by observation of several meiotic events and processes. Previous work in mice has shown that exposure to BPA in utero lead to aberrations during prophase of meiosis I including incomplete synapsis, end-to-end chromosome fusions, and an increased number of recombination foci corresponding to elevated recombination frequencies and altered genetic exchange distribution (Susiarjo et al., PLoS Genet. 2007; 3(1):e5. PMCID: 1781485). These defects in the mice may have resulted in increased chromosome non-disjunction as highlighted by the greater number of aneuploid eggs and embryos observed (Susiarjo et al., PLoS Genet. 2007; 3(1):e5. PMCID: 1781485; Hunt et al., Curr Biol. 2003; 13(7):546-53).

The correlation of these findings with the analysis in the mammalian systems, indicate that C. elegans can serve as a highly relevant assay system to identify agents that can function as reproductive toxicants in mammals (e.g., humans). In addition, this assay system can be used in high-throughput screening, to provide a fast and cost-effective means for the identification of environmental chemicals (individual and mixtures) that have detrimental effects on reproduction.

One aspect of the present invention relates to a method for assessing the biological safety of one or more test agents. The method comprises analyzing the test agent(s) for the ability to disrupt meiosis in C. elegans (e.g., by the methods disclosed herein). A determination that the test agent(s) disrupt meiosis indicates that the agent(s) has reduced biological safety to mammals (e.g., humans). The assessment is made by one or more of the methods described herein. A determination that the test agent can disrupt meiosis in C. elegans is an indication of reduced biological safety of that molecule.

In one embodiment, assessing the biological safety of one or more test agents comprises providing one or more test agents, and a culture of C. elegans, contacting the one or more test agents to the C. elegans within the culture, and analyzing meiosis in the C. elegans and comparison to an appropriate control to thereby determine the presence or absence of meiotic disruption. An increase in meiotic disruption of the C. elegans, compared to the control, indicates the test agent has reduced biological safety to mammals.

Due to the high conservation between C. elegans and mammals of the meiotic components, a positive identification of a test agent in the assays described herein indicates a significant potential exists for that agent to serve as a reproductive toxicant in mammalian systems. As such, the methods described herein can identify a likely mammalian reproductive toxicant. Once so identified, the agent can be further tested in mammalian systems for toxic effects.

In one embodiment, identifying a likely mammalian reproductive toxicant comprises providing one or more test agents, and a culture of C. elegans, contacting the one or more test agents to the C. elegans within the culture, and analyzing meiosis in the C. elegans and comparison to an appropriate control to thereby determine the presence or absence of meiotic disruption. An increase in meiotic disruption of the C. elegans, compared to the control, indicates the test agent is a likely mammalian reproductive toxicant.

The results of the assays described herein are relevant to all organisms that undergo meiosis, for example, vertebrate, invertebrate, avian, amphibian, reptile, mammal, primate (e.g., human), equine, bovine, porcine, feline, canine. An indication of meiotic disruption in C. elegans is an indication that the test agent has a strong likelihood of also causing meiotic disruption, and therefore serving as a reproductive toxicant, in such higher organisms. As such, the assay directly relates to assessing the biological safety of a test agent to such higher organisms.

### Analysis of Meiosis to Identify Meiotic Disruption

Meiotic disruption can take a variety of forms and as such meiotic disruption can be determined by a variety of methods. Disruption can result in a change in the duration of an examined meiotic process, it can be a change in the period of development in which a process takes place, it can be a delay or an acceleration of a process, an inefficiency of a process, or absence of a process. It can be a change in morphology, or any other observable/detectable difference, or it can be detected by observing a different outcome than would normally result from meiosis.

The C. elegans exposed to the test agent can be analyzed for disruption of meiosis by a variety of available means. All the classical stages of meiosis are present in a temporal/spatial gradient within the C. elegans gonad such that nuclei in each phase of meiosis are found in a specific region of the gonad. This makes the identification and analysis of the stages of meiosis straightforward. The effect of a potential toxicant on meiosis can be analyzed by using any combination of genetic, cellular and biochemical approaches. The analysis can take place at a specific stage, or at multiple stages, or throughout meiotic progression. In one embodiment, analysis is by assessing one or more meiotic markers (e.g., qualitatively and/or quantitatively). Such assessment can be done, for example by immunostaining with antibodies specific to such markers. Assessment can be for the pattern or staining, or for the intensity of staining (which is reflective of the quantity of the marker present), or a combination of the two.

Aberrant kinetics of DNA repair also indicates meiotic disruption. By observing nuclei immunostained for markers of DNA repair at different positions along the gonad, one can directly assess the levels of repair for individual nuclei, and also the kinetics of repair through the course of Prophase I.

In one embodiment, analysis is by monitoring the activation of a DNA damage checkpoint. This can be done, for example, by assaying for an increase in any DNA checkpoint components (e.g., ATL-1). An increase in the level of a DNA checkpoint component (e.g., ATL-1, in zones 6 and 7 of C. elegans which correspond to late pachytene) indicates the presence of meiotic disruption. In one embodiment, the number of germ cell apoptotic nuclei are monitored. An increase in germ cell apoptotic nuclei can indicate the activation of a DNA damage checkpoint and the presence of meiotic disruption.

In another embodiment, analysis is by monitoring the level of RAD-51 foci. Elevated levels, persistent levels or the appearance of large foci of RAD-51, a protein involved in strand invasion/exchange during double-strand break repair, (e.g., at the connection between the rest of the nucleus and a DAPI-stained chromosome fragment) indicates the presence of meiotic disruption.

In one embodiment, the analysis for meiotic disruption is by utilizing transgenic reporter lines and 3-D imaging of germline nuclei to assess changes in chromosome morphology. The oocyte position along the gonad and morphology of the nuclei indicate the exact stage of meiosis for a particular nucleus. In C. elegans, high-resolution 3-D imaging of meiotic chromosomes is performed in the context of an intact nuclear architecture. Relevant observations of aberrant chromosome morphology and/or oocyte positioning and/or nuclear morphology, indicate meiotic disruption by the test agent.

In one embodiment, analysis is by examination of chromosome morphogenesis throughout the germline of the worm. This can be accomplished by a straightforward whole worm fixation and DNA staining method (Colaiacovo et al., Genetics. 2002; 162(1):113-28. PMCID: 1462232). Visualization of chromosome morphogenesis in the germlines of exposed worms and their offspring, using microscopy and imaging tools allows for the identification of the steps of meiosis that are affected by the test agent. The culture can be assessed for chromosome morphology during diakinesis, wherein meiotic disruption is indicated by the observation or detection of aberrant chromosome morphology. Examples of such aberrant morphology include, without limitation, decondensed bivalents and chromatin bridges.

In one embodiment, meitoic disruption is analyzed by assessing the culture for disassembly of the synaptonemal complex at diakinesis. Meiotic disruption is indicated by observation or detection of aberrant disassembly of the synaptonemal complex anywhere between late pachytene and the end of diakinesis. One way this can be detected is by the detection of SYP-1 (a structural component of the synaptonemal complex) association with the chromosomes.

### Determination of an Increase in Percentage of Males

In one embodiment, the C. elegans is analyzed for disruption of meiosis by analyzing the percentage of males in the progeny of hermaphroditic worms. An increase in the percentage of males in the progeny population indicates an increase in disruption of meiosis. This can be, for example by determining the percentage of male adults, or the percentage of males at any other detectable stage of development. In one embodiment, the percentage of male embryos produced are quantitated. Quantitation of percentage of males at the embryonic stage can negate the possible effects of lethality on the results of the assay. Generally the percentage of males in a regular population is extremely low, about 0.1-0.2% (Hodgkin et al., Genetics. 1979 January; 91(1):67-94). However, meiotic disruption has been shown to increase this percentage by a significant amount. Without being bound by theory, it is thought that this is due to an increased incidence of the incorrect segregation or distribution of the sex chromosome during the meiotic cell division.

In one embodiment, the percentage of male embryos is assessed by determining the percentage of male embryos in adult mid-ventral sections. An increase in the percentage of male embryos (e.g., as compared to a standard or an appropriate control), indicates the existence of meiotic disruption. In one embodiment, the male embryos are identified by utilization of a reporter strain of C. elegans that contains a male specific nucleic acid reporter construct in expressible form. Such a construct can be generated, for example, by generating a nucleic acid construct that has a promoter from a male specific C. elegans gene, operably linked to a nucleic acid encoding a reporter protein. Such a reporter is a protein or polypeptide that is rapidly and specifically detectable. Such polypeptides are referred to in the art as detectable markers. Generally detection is by specific binding to a protein or other factor, or by chemical and/or visual means. In one embodiment, the reporter is a molecule that is visually detectable.

A number of male specific genes are know in the art and can be used in the methods disclosed herein. A male specific gene that is expressed early in development will allow identification of males at that earlier period of development. In one embodiment, the gene is xol-1. In one embodiment, the male specific nucleic acid reporter construct is xol-1-GFP. The xol-1-GFP already introduced into C. elegans in expressible form is found in the xol-1::GFP reporter strain of C. elegans (Kelly et al., Genetics 156: 617-30 (2000)).

In addition to the male specific nucleic acid reporter construct, the C. elegans may optionally also contain one or more other nucleic acid reporter constructs in expressible form. In one embodiment, the other nucleic acid reporter construct is ubiquitously expressed in the C. elegans. Detection of such a ubiquitously expressed construct can be used, for example, to correct for lethality or for slow growth of the organism.

The experiments detailed herein present the first use of an increase in the male population to indicate meiotic disruption by an agent to which the C. elegans is externally exposed, other than ethyl methane sulfonate (previously used as a powerful mutagen to generate genetic mutants for meiotic disruption in C. elegans). Prior to this report, the analysis had been used for the generation and identification of genetic mutans of meiosis. As such, another aspect of the invention relates to a method for assessing one or more test agents for the ability to disrupt meiosis in C. elegans. The method comprises assessing for the ability of one or more test agents to cause an increase in the percentage of male progeny in the C. elegans. The identification of such ability indicates that the test agent disrupts meiosis in the C. elegans. In one embodiment, the methods comprises providing one or more test agents, and a culture of C. elegans, contacting the one or more test agents to the C. elegans within the culture, and determining the percentage of male embryos in adult C. elegans mid-ventral sections. An increase in the percentage of male embryos, compared to an appropriate control, indicates the agent disrupts meiosis in C. elegans. In one embodiment, the various methods described herein omit the use of ethyl methane sulfonate (EMS) as a test agent. In one embodiment, the test agent used in the various methods described herein is not a known mutagen.

### Detectable Markers

The present invention contemplates the use of detectable markers in the identification of male C. elegans. In one embodiment the detection is by detection of a fluorescence or luminescence from a detectable marker. In one embodiment, the detectable marker is a fluorescent or luminescent protein as it allows for longitudinal studies on live animals, although other markers such as beta-galactosidase, enzymes that react to produce colored reaction products, may also be used. One major class of detectable marker genes that may be used to visualize a live C. elegans worm in its intact form comprise the fluorescent proteins such as the green fluorescent proteins (GFP). GFP is a naturally fluorescent protein which can be used to mark the cells in which a promoter is active. It has the benefit that the animals can be observed live.

The GFP proteins, originally isolated from the jellyfish Aequorea victoria retain their fluorescent properties when expressed in heterologous cells thereby provides a powerful tool as fluorescent recombinant probes to monitor cellular events or functions. Several spectral and mutational variants of GFP proteins have been isolated, for example, the naturally occurring blue-fluorescent variant of GFP (Heim et al. 1994; U.S. Pat. No. 6,172,188, both incorporated herein by reference), the yellow-fluorescent protein variant of GFP (Miller et al., 1999), the red fluorescent protein isolated from the coral Discosoma (Fradkov et al., 2000; Miller et al., 1999), which allows the use of fluorescent probes having different excitation and emission spectra permitting the simultaneous monitoring of more than one process. GFP proteins provide non-invasive assays which allow detection of cellular events in intact, living cells. The skilled artisan will recognize that the invention is not limited to the fluorescent proteins described and one may use any other spectral variant or derivative.

Luciferase is another useful detectable marker. Luciferase is a generic term for the class of oxidative enzymes used in bioluminescence. In one embodiment, the luciferase is from the firefly Photinus pyralis.

Suitable detectable marker molecules include autonomous fluorescent proteins (AFPs) such as green fluorescent protein (GFP) and blue fluorescent protein (BFP), aequorin, alkaline phosphatase, luciferase, β-glucuronidase, β-lactamase, β-galactosidase, acetohydroxyacid synthase, chloramphenicol acetyltransferase, horseradish peroxidase, nopaline synthase or octapine synthase.

Detectable markers such as β-galactosidase, enzymes such as such as urease, alkaline phosphatase, and horseradish peroxidase, can be detected either spectrophotometrically by the used of calorimetric indicator substrates. These markers are somewhat limited in their use as they cannot be used to monitor live worms and worms must be killed and fixed to visualize detection. In the case of β-galactosidase, lacZ is a stable enzyme, with negligible background activity in C. elegans. The chromogenic stain is stable, and the enzyme is still active after mild fixation. Generally, a lacZ with a nuclear localization signal, derived from SV40, is used so that the nuclei of cells expressing the construct can be identified.

It is contemplated that one or more detectable markers may be used at one time, for monitoring or control purposes. The use of multiple markers can contribute to high-throughput screening. In one embodiment, two different luciferase molecules, which can be independently and simultaneously detected, are used a detectable markers. In one embodiment, the two different luciferase molecules are firefly luciferase and Renilla luciferase.

A positive indication of meiotic disruption by one of the assays described herein, is, for example, a reproducible, statistically significant deviation from normal meiosis. Normal meiosis is determined or represented by comparable analysis of an appropriate control (e.g., a control population). Normal meiosis can also be determined or represented by a pre-established standard.

### Controls

In some embodiments, the various analyses described herein are compared to an appropriate control to determine meiotic disruption. An increase in meiotic disruption of the C. elegans, compared to the control, indicates induction of meiotic disruption by the test agent. One example of an appropriate negative control is an identical C. elegans or population, treated identically, other than exposure to the test agent(s). The populations are as identical as is determined necessary experimentally to provide an adequate control. Generally a control C. elegans or population will be of the identical genetic makeup to the test organism or population. In one embodiment, the control is of the same age as the test organism or population.

### Synchronization of Cultures

In one embodiment, the screening methods described herein are performed using synchronized worm cultures. Synchronized worms are worms that are in the same growth stage. The various growth stages of nematode worms such as C. elegans are eggs, the L1 stage, L2 stage, L3 stage, L4 stage and adult stage.

In one embodiment, synchronization is by hypochloride (bleach) treatment of the worm population. Such treatment kills all the adult worms and larvae, while the embryos are protected from the bleach by the egg shell. Synchronization allows a rapid assessment of whether a test agent causes a delay in development.

Other techniques suitable for use in generating the various synchronized cultures are known in the art, see for example Methods in Cell Biology, vol 48, Ed. H. F. Epstein and D. C. Shakes, Academic Press. The main population of a standard C. elegans culture consists of hermaphrodite worms, so it does not require special techniques to generate synchronized hermaphrodite nematodes in different growth stages.

In one embodiment, the synchronized nematode worms are of a specific sex. The synchronized cultures can be hermaphrodites or males or nematodes in special larval stage, designated dauers. To generate male worms, several techniques have been described in the literature. C. elegans cultures that are enriched or consist exclusively of male worms, have been described in C. elegans II, ed, By Fiddle, Blumenthal, Meyer and Pries, 1997, CSHL press. Strains for making enriched or pure male samples have been described by Jonathan Hodgkin, Worm Breeder's Gazette 15(5):19, 1999).

### Other Organisms

The skilled practitioner will recognize that the assay methods described herein for performance in C. elegans can be performed using any other nematode worms from the genus Caenorhabditis, e.g., C. briggsae. It will further be appreciated that the screening methods described herein could be carried out with other nematodes and in particular with other microscopic nematodes. As used herein the term “microscopic” nematode encompasses nematodes of approximately the same size as C. elegans, being of the order 1 mm long in the adult stage. Microscopic nematodes of this approximate size are extremely suited for use in mid- to high-throughput screening as they can easily be grown in the wells of a multi-well plate. As such, the methods described herein are also intended to encompass use of such alternate organisms in place of C. elegans, as described.

### Candidate Agents for Screening

The present invention comprises methods for screening a variety of substances for meiotic disruption activity. These assays may comprise random screening of large libraries of candidate substances; alternatively, the assays may be used to focus on particular classes of compounds selected for reasons of suspicion of being meiotic disruptors (e.g., structural attributes). Such libraries include, without limitation, libraries from the National Toxicology Program, each with ˜1408 environmentally relevant compounds, which includes toxicants with described reproductive toxicity as well as many known endocrine disruptors (Boyd et al., Neurotoxicol Teratol. 2010; 32(1):68-73; Cho et al., Toxicol In Vitro. 2008; 22(4):1099-106. PMCID: 2386563; Xia et al., Environ Health Perspect. 2008; 116(3):284-91. PMCID: 2265061), and the ToxCast 320 library of pesticide actives identified by the US EPA (Dix et al., Toxicol Sci. 2007; 95(1):5-12).

### Administration of the Agents

The C. elegans is exposed to the test agent such that the test agent is taken up by the organism. Uptake can be, for example, through the cuticle, by ingestion (oral), or by any other means of entry into the body from the surrounding media. For example, the test agent is mixed with the media in which the organisms is grown/cultured. Following a predetermined amount of time of exposure, the C. elegans is then analyzed for disruption of meiosis by the test agent by the methods described herein. The addition of test compounds to the media of C. elegans is discussed in Rand and Johnson in Methods of Cell Biology, Chapter 8, volume 48, Caenorhabditis elegans: Modern Biological Analysis of an Organism Ed. Epstein and Shakes, Academic Press, 1995 and J. Ahringer in Curr. Op. in Gen. and Dev. 7, 1997, 410-415.

In one embodiment of the methods described herein, the test agent is assessed at concentrations that are physiologically relevant to mammalian systems. Such concentrations can be determined by the skilled practitioner for each specific test agent examined. In one embodiment, the test agent is assessed in concentrations routinely found, or expected to be found, in mammalian tissue or systems (e.g., from routine exposure). In another embodiment, the test agent is assessed in concentrations routinely found in the environment. In one embodiment, the test agent is assessed at from about 1 nM to about 1 mM. In one embodiment, the test agent is assessed at from about 1 μM to about 100 mM. In one embodiment, the test agent is assessed at from about 100 μM to about 1 mM. Useful concentrations for assessment of a test agent, include without limitation, about 1 μM, about 5 μM, about 10 μM, about 20 μM, about 50 μM, about 80 μM, about 100 μM, about 200 μM, about 500 μM, about 750 μM, about 0.1 mM, about 0.5 mM, about 1 mM, about 5 mM, about 10 mM, about 20 mM, about 50 mM, about 75 mM, and about 100 mM. In one embodiment, the test agent is assessed at three different concentrations. In one embodiment, the test agent is assessed at about 1 μM, about 100 μM, and about 1 mM.

Higher and lower concentrations may also be useful, depending upon the specific test agent being assessed. Higher concentrations may be used, e.g., for test agents that have limited to poor solubility. The test agent may be dissolved in a solvent prior to the exposure to facilitate uptake by the C. elegans.

A determination of the assessment concentration can be either by quantitation of the amount contacted to the C. elegans, or by quantitation of the amount actually delivered internally to the C. elegans. The amount delivered internally to the C. elegans can be determined, for example, by measuring uptake and determining the weight of agent/g of worm extract (e.g., measuring internal concentrations of free test agent by tandem HPLC).

The test agent may be dissolved in a solvent prior to the exposure to facilitate uptake by the C. elegans.

The C. elegans can be exposed to the test agent(s) at any phase in the growth cycle. In one embodiment, exposure to the C. elegans begins at the egg stage. In one embodiment exposure of the C. elegans begins during L1 (about 12 hours after the egg stage). In one embodiment exposure of the C. elegans begins during L2. In one embodiment exposure of the C. elegans begins during L3. In one embodiment exposure of the C. elegans begins during L4. Exposure which begins during a specified stage can occur, for example at the beginning of that stage, or anytime throughout that stage (e.g., from about 1, 2, 3, 4, . . . 13, or about 14 hours into the stage). In one embodiment exposure of the C. elegans begins once L4 is complete (referred to as post-L4). In one embodiment, exposure begins at a time point within the range of about 1 to about 17 days post L4. In one embodiment, exposure begins at a time point within the range of about 2 to about 17 days post L4 (e.g., at about 2, 3, 4, 5, . . . 15, 16, or about 17 days post L4).

The duration of exposure of the test agent to the C. elegans can also be varied for the methods described herein. Exposure can range from less than a day (e.g., from about 23 hr, about 22 hr, about 21 hr, . . . down to about 1 hr) to the entire duration of life which is about 21 days. Shorter periods of exposure are also envisioned (e.g., from about 20 days, about 19 days, about 18 days, about 17 days . . . , to about 1 day). In one embodiment, exposure is for about 4 days.

In one embodiment, the exposure begins at the egg stage (day 0), and is for about 4 days, at which time the C. elegans are analyzed for meiotic disruption. In one embodiment, the exposure begins at the egg stage (day 0), and is for about 5 days, at which time the C. elegans are analyzed for meiotic disruption. In one embodiment, the exposure begins at the egg stage (day 0), and is for about 6 days, at which time the C. elegans are analyzed for meiotic disruption. In one embodiment, the exposure begins at the egg stage (day 0), and is for about 7, 8, 9, . . . 20 or 21 days, at which time the C. elegans are analyzed for meiotic disruption.

If exposed to more than one test agent, exposure can be at the same time, or can be at different times in the growth cycle. The same or different durations of exposure can also be used for co-exposure to the various test agents. Furthermore, the test-agents co-administered can be administered at the same or different concentrations. Depending upon the specific agents, it may be necessary or beneficial to dissolve one or more test agents in a solvent. It may be useful to dissolve different agents in different solvents.

### C. Elegans Mutants and Transgenics

Although the skilled practitioner will recognize that most of the methods described herein can be performed with wild type C. elegans, it may be useful to use C. elegans that contain one or more genetic mutations to facilitate the methods. In one embodiment, the genetic mutation is a mutation that affects the integrity of the C. elegans cuticle. Such mutations include, without limitation, bis-1(nx3), dpy-2(e8), dpy-7(e88) and dpy-10(e128) (Watanabe et al., Mutation Research 570: 71-80 (2005). A variety of such mutations, and their generation in the appropriate C. elegans backgrounds (e.g, by crossing) can be achieved by the skilled practitioner.

Alternatively, or in addition, it may be useful to use C. elegans that contain one or more transgenes to facilitate the methods. For example, the transgene can be a reporter construct to facilitate detection of one of the indications of meiotic disruption described herein. Routine methods for the construction of transgenic C. elegans are well known in the art (Methods of preparing transgenic worms are well known in the art and are particularly described by Craig Mello and Andrew Fire, Methods in Cell Biology, Vol 48, Ed. H. F. Epstein and D. C. Shakes, Academic Press, pages 452-480) and with the use of appropriate promoter sequences transgenic C. elegans can be constructed which express a genetically encoded marker molecule in all cells, in a particular tissue or in one or more specified cell types.

In one embodiment, a collagen deficient mutation for increased sensitivity is present in the same context as is a reporter construct for detection of male specific embryos. For example, such an organism can be generated by crossing the xol-1:GFP (located on chromosome V) or xol-1:luciferase reporter with the bis-1 allele (located on chromosome IV). Such a cross generates an organism that has increased sensitivity to exogenously added agents, and would facilitate a high-throughput screening of test agents that have reduced solubility.

### Culture of C. Elegans

In one embodiment, the assays described herein are performed on C. elegans cultured in liquid medium. In one embodiment, the assays described herein are performed on C. elegans cultured on solid or semi-solid medium. It may be beneficial to add or omit certain components to the medium to facilitate the methods described herein. Such components omitted or reduced would be, for example, those which might otherwise mask the effects of a test agent. Such components added or increased would be, for example, those which might increase the sensitivity of the C. elegans to the test agent (e.g., facilitate entry of the test agent into the organisms). In one embodiment, cholesterol in the medium is reduced or omitted. This can facilitate detection of the effects of certain steroid like test agents (Tominaga et al., Journal of Health Science 49: 28-33 (2003). Without being bound by theory, it is though that cholesterol is required for the synthesis of steroid hormones and removing it from the medium challenges the worm hormonally and this allows the determination of external hormonal effects.

### High-Throughput Screening Assay

The methods of the invention can be performed in a multi-well plate format and are therefore particularly suitable for use in mid-to-high throughput screening. In a preferred embodiment, the multi-well plates have 96 wells, but the invention is also applicable to multi-well plates with another number of wells, which include but is not restricted to plates with 6, 12, 24, 384, 864 or 1536 wells. The terms “multi-well plate” and “microtiter plate” are used interchangeably throughout.

In order to generate quantitative results using the methods of the invention it may be important to ensure that substantially equal numbers of individual nematodes are added to each of the wells. The precise number of worms added to the wells may vary depending upon the type of screen being performed and the required sensitivity. The methods described herein using multiwell plates (e.g., for high-throughput screening, such as 96 well plates), about 1 to 100 worms per well are used, for example, about 10 to 80 worms per well are used. In one embodiment, from about 25 to about 30 worms per well are used. In one embodiment, about 25 worms per well are used.

Various methods can be used to ensure that substantially equal numbers of worms are added to each of the wells. One way in which this can be achieved is by taking worms cultured according to the standard procedures known to those skilled in the art in solid or liquid media and re-suspending the worms in a viscous solution to form a homogeneous suspension. The viscosity of the solution maintains an even distribution of worms in the suspension, thus substantially equal numbers of worms can be dispensed by adding equal volumes of the homogeneous worm suspension to each of the wells. Suitable viscous solutions include a solution containing a low concentration of a polymer material (e.g. 0.25% low melting point agarose), glycerol etc.

As an alternative to the above-described approach an equal distribution of worms over the wells of the multi-well plate can be achieved using a worm dispensing device, such as that developed by Union Biometrica, Inc. The worm dispenser can be programmed to add a set number of worms to each of the wells of the plate. In addition, it can be used to select worms in such a way that only hermaphrodites or males or dauers are dispensed and it can also select on the basis of size so that specifically eggs, L1, L2, L3, L4 or adult worms are dispensed.

In one embodiment, the assays described herein are performed in liquid medium. In one embodiment, a water soluble polymer is included in to the medium in order to increase its viscosity, such as that described in U.S. Patent Application 2003/0154501.

A number of other such liquid culture mediums designed for high-throughput screening are known and available in the art, and can be adapted for the methods described herein by the skilled practitioner.

In one embodiment of the invention, synchronized cultures of worms are grown for the desired developmental period (e.g., from eggs to adulthood, 4 days) on multiwell plates (e.g., 96-well plates) containing media mixed with the compound of interest at the concentration of interest (e.g., 1 μM and 100 μM). The concentrations can be adjusted if necessary to compensate for solubility differences. Following exposure, the worms are washed off the plate with a physiological buffer and then run through a high-throughput flow cytometry device (e.g., the COPAS Biosort). With this automated nematode analysis, sorting, and dispensing platform (Furlong et al., Nat Biotechnol. 2001; 19(2):153-6; Gill et al., Free Radic Biol Med. 2003; 35(6):558-65; Rea et al., Nat Genet. 2005; 37(8):894-8. PMCID: 1479894) one 96-well plate can be screened per hour. In one embodiment, test wells are alternated with mock wells to flush the equipment in order to avoid cross-contamination. This allows screening of ˜40 wells/hour in practice. The worm sorter not only directs the sorting of GFP positive worms from GFP negative ones but when coupled to the Profiler analysis software, also allows for the localization of the fluorescence within the worm. In one embodiment, scoring is for GFP positive eggs present in the animal's mid-ventral section (uterus), therefore, any worms showing mis-localized GFP expression will be discarded thus improving the accuracy of the screen. Positive controls used for the screen can include colchicine and nocodazole, both described as disruptors of spindle organization and therefore of chromosome segregation (Sato A, et al., Cell. 2009; 139(5):907-19; Stear et al., Mol Biol Cell. 2004; 15(11):5187-96. PMCID: 524797). Preliminary analysis of these controls revealed the following frequencies of xol-1::GFP worms containing GFP+ eggs: 0/50 exposed to vehicle (1% DMSO), and 6/16 and 3/66 exposed to 0.1 mg/ml nocodazole and demecolcine, respectively.

Identification of Agents that Affect Fat Homeostasis

The assays described herein can also be used to identify chemicals that disrupt fat homeostasis. Because fat metabolism pathways are highly conserved between the worm and humans, and C. elegans is increasingly viewed as a powerful model to understand the genetic pathways regulating fat synthesis and adiposity (Schlegel and Stainier, 2007; Watts, 2009). Such disruptors so identified by the methods described herein have reduced biological safety to mammals in that they have a significant potential to disrupt fat homeostasis in higher organisms, (e.g., mammals, especially humans).

One aspect of the invention relates to a method for identifying a likely disruptor of fat homeostasis in a mammal. The method comprises contacting one or more test agents to a culture of C. elegans and analyzing the culture for fat content. A change in the fat content, as compared to an appropriate control, indicates that the test agent(s) is a likely disruptor of mammalian fat homeostasis. In one embodiment, analyzing is by staining the C. elegans with oil red O to stain the fat. In one embodiment, the change in fat content is selected from the group consisting of an increase in fat content, a decrease in fat content, and a redistribution of fat content.

The method involves administration of the test agents to thereby contact the C. elegans, as described herein, and then determination of fat content of the C. elegans. A change in the fat content (e.g., an increase or decrease), for example, by comparison to an appropriate control, will indicate that the test agent disrupts fat homeostasis in C. elegans. In one embodiment the method comprises providing one or more test agents, and a culture of C. elegans, contacting the one or more test agents to the C. elegans within the culture, and analyzing fat homeostasis in the C. elegans and comparison to an appropriate control to thereby determine the presence or absence of fat homeostasis disruption. An increase in fat homeostasis disruption of the C. elegans, compared to the control, indicates the test agent has reduced biological safety to mammals and has significant potential to disrupt fat homeostasis in higher organisms.

In one embodiment, fat content is detected in C. elegans by staining the worms with various chemicals including oil red O (Kimura et al, 1997; Ashrafi et al, 2003, O'Rourke et al, 2009). In one embodiment, the C. elegans are exposed for 4 days to the test agent (e.g., from an environmental compounds library) and then treated overnight with oil red O for staining of the fat (as described in O'Rourke et al, 2009). The fat will stain red and the intensity of red staining can be captured and analyzed with a regular light microscope coupled to a color camera and computer. A positive indication of fat homeostasis disruption includes, without limitation, an effect on (1) the intensity of red staining and (2) the localization of the red staining (redistribution of fat storage). A detected increase in the intensity of the red staining indicates more fat is being stored, and a detected decrease in fat staining indicates that less fat is being stored. Any determined difference from an appropriate control indicates that the test agent is a disruptor of fat homeostasis.

A positive indication of fat homeostasis disruption by one of the assays described herein, is, for example, a reproducible, statistically significant deviation from normal fat homeostasis. Normal fat homeostasis is determined or represented by comparable analysis of an appropriate control (e.g., a control population). Normal fat homeostasis can also be determined or represented by a pre-established standard.

In certain aspects of the present invention, all the necessary components for conducting the assays described herein may be packaged into a kit. Such kits, including any instructions contained therein for the use of the contents in the methods described herein, are encompassed in the invention.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used to describe the present invention, in connection with percentages means ±1%.

In one respect, the present invention relates to the herein described compositions, methods, and respective component(s) thereof, as essential to the invention, yet open to the inclusion of unspecified elements, essential or not (“comprising). In some embodiments, other elements to be included in the description of the composition, method or respective component thereof are limited to those that do not materially affect the basic and novel characteristic(s) of the invention (“consisting essentially of”). This applies equally to steps within a described method as well as compositions and components therein. In other embodiments, the inventions, compositions, methods, and respective components thereof, described herein are intended to be exclusive of any element not deemed an essential element to the component, composition or method (“consisting of”).

All patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The present invention may be as defined in any one of the following numbered paragraphs.
1. A method for assessing the biological safety of one or more test agents, comprising, contacting the one or more test agents to a culture of C. elegans and analyzing the culture for meiotic disruption, wherein an increase in meiotic disruption of the C. elegans, indicates that the test agent(s) has reduced biological safety to mammals.
2. A method to identify a likely mammalian reproductive toxicant, comprising contacting the one or more test agents to a culture of C. elegans and analyzing the culture for meiotic disruption, wherein an increase in meiotic disruption of the C. elegans, indicates the test agent(s) is a likely mammalian reproductive toxicant.
3. A method for assessing the biological safety of one or more test agents, comprising,
   a) providing one or more test agents, and a culture of C. elegans,
   b) contacting the one or more test agents to the C. elegans within the culture, and
   c) analyzing meiosis in the C. elegans and comparison to an appropriate control to thereby determine the presence or absence of meiotic disruption;
      wherein an increase in meiotic disruption of the C. elegans, compared to the control, indicates the test agent has reduced biological safety to mammals.
4. A method to identify a likely mammalian reproductive toxicant, comprising
   a) providing one or more test agents, and a culture of C. elegans,
   b) contacting the one or more test agents to the C. elegans within the culture, and
   c) analyzing meiosis in the C. elegans and comparison to an appropriate control to thereby determine the presence or absence of meiotic disruption;
      wherein an increase in meiotic disruption of the C. elegans, compared to the control, indicates the test agent is a likely mammalian reproductive toxicant.
5. The method of paragraph 3 or 4, wherein analyzing meiosis is by assessing the culture for % male embryos in adult midventral sections, and meiotic disruption is indicated by an increase in % male embryos.
6. The method of paragraph 3 or 4, wherein analyzing meiosis is by assessing the culture for number of apoptotic nuclei, for RAD-51 foci, or levels of ATL-1, in adult germlines at zones 6 and 7 at late pachytene, and meiotic disruption is indicated by an increase in RAD-51 foci, an increase in the number of apoptotic nuclei or an increase in the level of ATL-1.
7. The method of paragraph 3 or 4, wherein analyzing meiosis is by analyzing the culture for chromosome morphology during diakinesis, and meiotic disruption is indicated by aberrant chromosome morphology during diakinesis.
8. The method of paragraph 3 or 4, wherein analyzing meiosis is by analyzing the culture for disassembly of the synaptonemal complex and meiotic disruption is indicated by aberrant disassembly of the synaptonemal complex at from about late pachytene to about the end of diakinesis.
9. A method for assessing one or more test agents for the ability to disrupt meiosis in C. elegans, comprising:
   a) providing one or more test agents, and a culture of C. elegans,
   b) contacting the one or more test agents to the C. elegans within the culture,
   c) determining the percentage of male embryos in adult mid-ventral sections;
      wherein an increase in the percentage of male embryos, compared to an appropriate control, indicates the agent disrupts meiosis in C. elegans.
10. The method of paragraph 5 or 9, wherein the C. elegans contains a male specific nucleic acid reporter construct in expressible form.
11. The method of paragraph 10, wherein the nucleic acid reporter construct comprises a promoter from a male specific gene, operably linked to a nucleic acid encoding a reporter protein.
12. The method of paragraph 11, wherein analyzing comprises detecting an increase in the reporter protein as compared to an appropriate control.
13. The method of paragraph 11, wherein the gene is xol-1.
14. The method of paragraph 11, wherein the reporter is selected from the group consisting of GFP and luciferase.
15. The method of paragraph 10, wherein the male specific nucleic acid reporter construct is xol-1-GFP.
16. The method of paragraph 10, wherein the C. elegans further contains a ubiquitously expressed reporter construct.
17. The method of paragraphs 1-16, wherein the culture of C. elegans is grown in a liquid culture medium designed for high-throughput screening.
18. The method of paragraph 17, wherein the C. elegans culture comprises about 25 worms/well, in one or more wells of a 96 well plate.
19. The method of paragraphs 1-18, wherein the agent(s) is contacted to the culture by incorporation into the growth medium, at a concentration of from about 1 μM to about 100 mM.
20. The method of paragraphs 1-19, wherein the agent(s) is contacted to the C. elegans for a duration of about 1-6 days.
21. The method of paragraphs 1-20, wherein the agent(s) is contacted to the C. elegans for a duration of about 4-6 days.
22. The method of paragraphs 1-21, wherein the agent(s) is contacted to the C. elegans for a duration of about 4 days.
23. The method of paragraphs 1-22, wherein the agent(s) is first contacted to the C. elegans at the developmental stage ranging from eggs to 17 days post L-4.
24. The method of paragraphs 1-23, wherein the agent is first contacted to the C. elegans at a developmental stage from eggs to 2 days post-L4.
25. The method of paragraphs 1-24, wherein the agent(s) is first contacted to the C. elegans as an egg.
26. The method of paragraphs 1-24, wherein the agent(s) is first contacted to the C. elegans at 2 days post-L4.
27. The method of paragraphs 1-26, wherein the agent(s) is dissolved in a solvent prior to addition to the C. elegans medium.
28. The method of paragraphs 1-27, wherein the C. elegans of the culture has a mutation that affects the integrity of its cuticle.
29. The method of paragraph 28, wherein the cuticle mutation is selected from the group consisting of bis-1(nx3), dpy-2(e8), dpy-7(e88) and dpy-10(e128).
30. The method of paragraphs 1-29, wherein a plurality of agents are co-assessed in a high-throughput assay.
31. The method of paragraph 30, wherein the assessment is made using flow cytometry.
32. A method for identifying a likely disruptor of fat homeostasis in a mammal, comprising, contacting one or more test agents to a culture of C. elegans and analyzing the culture for fat content, wherein a change in the fat content, as compared to an appropriate control, indicates that the test agent(s) is a likely disruptor of mammalian fat homeostasis.
33. The method of paragraph 1, wherein analyzing is by staining the C. elegans with oil red O to stain the fat.
34. The method of paragraph 1, wherein the change in fat content is selected from the group consisting of an increase in fat content, a decrease in fat content, and a redistribution of fat content.

The present invention is further illustrated by the following Examples. These Examples are provided to aid in the understanding of the invention and are not construed as a limitation thereof.

### EXAMPLES

C. elegans is a relevant model system for the study of meiosis as many cellular and molecular pathways are evolutionarily conserved between the worm and mammals (Kaletta et al., Nat Rev Drug Discov. 2006; 5(5):387-98; Leung et al., Toxicol Sci. 2008; 106(1):5-28. PMCID: 2563142). Indeed, genetic screens performed in the nematode have led to a dramatic increase in our understanding of the genes involved in the regulation of meiosis in mammals (reviewed in Colaiacovo M P. Chromosoma. 2006; 115(3):195-211; Pawlowski et al., Trends Cell Biol. 2005; 15(12):674-81).

### Example 1

### Effects of BPA on the C. Elegans Germline

Meiosis is the cell division program by which haploid sperm and eggs are generated from diploid germ cells and it is therefore essential for both sexual reproduction and generating genetic diversity. Errors in chromosome segregation during meiosis can result in aneuploidy which significantly contributes to infertility, miscarriages and birth defects in humans [1]. Superimposed onto the natural variations in human fertility, there is also an unequivocal contribution of environmental exposures to the etiology of human aneuploidies [2, 3].

Despite the devastating outcomes stemming from impaired meiosis, no alternatives to the slow, costly and technically challenging use of mammalian species for in vivo studies of environmental disruption of meiosis currently exist. Furthermore, there has been a tremendous interest in developing reliable systems of mammalian relevance that could ultimately be used in high-throughput screening strategies [4].

The use of the free-living soil nematode and genetic model Caenorhabditis elegans to dissect the action of Bisphenol-A (BPA) on the germline was explored. BPA is one of the highest production volume chemical with a global annual production scale approximating 4 million metric tons [5]. It is commonly used in the manufacturing of several polymers including polycarbonate and epoxy resins [6, 7] and is therefore found in plastic bottles, the lining of food and beverage cans and dental sealants among others [6, 7]. Consistent with its widespread presence, urinary BPA is detectable in over 90% of the US population from infants to adults [7-9] and higher levels are correlated with cardiovascular diseases, diabetes [9] as well as sexual dysfunction in men [10]. In rodent models of BPA exposure, multiple levels of reproductive impairments have been described including abnormal behavior, feminization of the male conceptus, decrease sperm production, altered estrous cycle and female reproductive tract (reviewed in [11, 12]).

Recently, BPA has been reported to impede meiotic progression in mice at two key steps, during prophase and metaphase of meiosis I [13, 14]. Specifically, in utero exposure to BPA at embryonic stages where prophase I occurs causes severe meiotic aberrations [14]. These include incomplete synapsis (the physical connection between homologous chromosomes), abnormal chromosomal configurations such as chromosome fusions and an increased number of recombination events. These defects ultimately result in increased chromosome nondisjunction as highlighted by the greater number of aneuploid eggs and embryos observed [14]. Although BPA has a severe impact on the mammalian meiotic program, we still lack in our understanding of the affected genes and pathways and mechanism of action in the germline.

To investigate how BPA exerts its effect on the germline, the numerous features that C. elegans offers for the study of meiosis were exploited. These include short reproduction cycle, large number of germline nuclei and a characteristic distribution of these nuclei in a spatio-temporal fashion allowing for easy access and identification of all stages of prophase I and. Most importantly, C. elegans displays a remarkable degree of gene conservation with humans [15, 16] which has proven invaluable for the isolation of genes pertinent to mammalian meiosis (reviewed in [17-22]). The following is a detailed analysis of the action of BPA on the maintenance of the germline in C. elegans. BPA is shown to have multiple effects on the germline: it decreases the size of the mitotic pool as well as impairs meiotic progression. During pachytene, BPA exposure leads to synapsis and chromosomal aberrations and an altered kinetics of double-strand DNA breaks (DSBs) repair leading to activation of checkpoint kinases ATL-1 and CHK-1 and apoptosis. These defects are correlated with an impaired maturation of the chromosomes in late prophase I and oocyte and early embryonic chromosomal abnormalities. Interestingly, both effects of BPA on the mitotic pool and late prophase can be rescued by co-exposure to estrogen indicating an anti-estrogenic action of BPA on the germline. A model of action is proposed whereby BPA, through a dramatic alteration of the meiotic DNA repair machinery, leads to failure to maintain proper chromatin and synaptic integrity.

### Results

### BPA Exposure Causes Sterility in C. Elegans

Exposure to Bisphenol A has been associated in several animal species with a variety of reproductive impairments including sex hormone imbalance [29-31], decreased quality and production of both sperm and oocytes [13, 14, 32-35], decreased embryonic viability and increased recurrence of miscarriages in humans [32, 36].

C. elegans is a useful system in which to perform assays for reproductive toxicants since the organization of Prophase I in the C. elegans gonad is readily visualized in DAPI-staining chromosomes through the C. elegans germline. Nuclei are positioned throughout the germline in a temporal/spatial gradient representing the various stages of meiotic prophase. Changes in the appearance of DAPI-stained chromosomes allow for prompt identification of these stages.

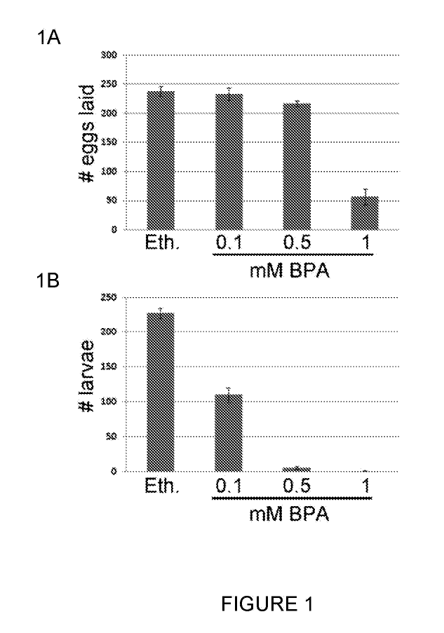

To address whether BPA also affects C. elegans reproduction, worms were exposed to various concentrations of BPA under several culture conditions. The parameters tested were: BPA concentration (100 uM, 500 uM or 1 mM), vehicle (DMSO or ethanol), cholesterol concentration (5 ug/ml, 0.5 ug/ml or none) and culture method (liquid or plate). The exposure of worms to BPA dissolved in 100% ethanol on plates without cholesterol, at a final concentration of 1 mM for 4 days (from eggs to adulthood), gave the most consistent results with low overall toxicity, as judged by growth and behavior of the worms. Using these conditions, a significant 4-fold reduction in the number of eggs laid (brood size) by worms exposed to 1 mM BPA, compared to vehicle (p=1.5.10⁻⁵, n=5) (FIG. 1A), was observed. Furthermore, embryonic viability was also dramatically reduced to 1.5% of controls (p=6.8.10⁻⁶, n=5) (FIG. 1B).

As a mutational screen previously identified several collagen proteins as mediators of BPA sensitivity [37], it was reasoned that the worm cuticle might act as a partial barrier to BPA and that internal BPA levels might be significantly lower. The internal concentration of free BPA was measured by tandem HPLC analysis (Axys, BC, Canada) following an extensive number of physiological buffer washes and lysis of worms cultured on BPA plates (Materials an Methods). Uptake of BPA was measured to be at 2 ug/g of worm extract, a value within the range of internal concentrations of BPA detected in several rodent exposure models [38-40] and of serum levels in occupational exposure cases [41]. It is also only an order of magnitude higher than the BPA levels found in human placental tissues [40, 42]. Taken together, these results demonstrate that BPA impairs C. elegans fertility at internal levels within the range of concentrations previously described in mammals.

### BPA Decreases the Size of the Mitotic Pool in the Premeiotic Region in the Germline

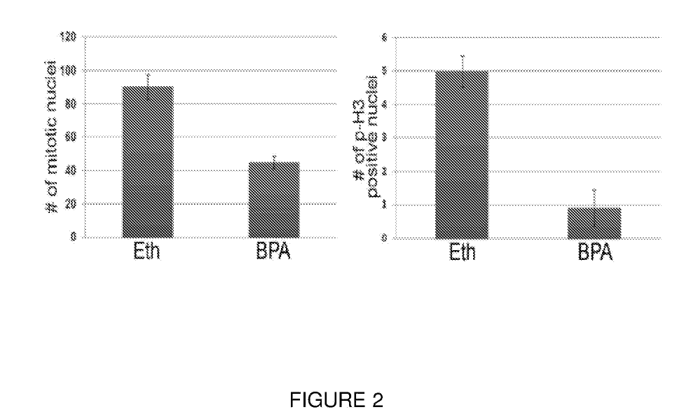

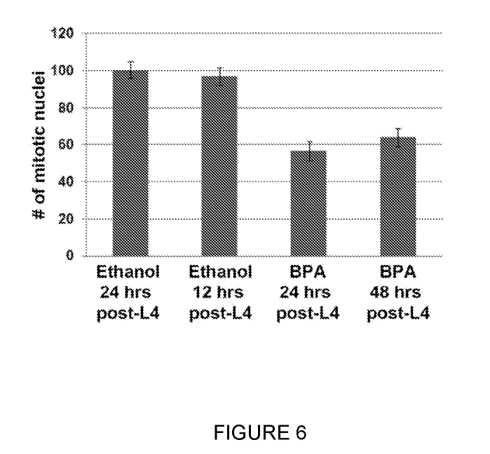

The observed decrease in embryonic viability suggests earlier defects taking place during oogenesis. BPA exposed worms were observed to have significantly smaller gonads compared to control worms, the number of dividing germline nuclei in BPA treated worms was examined. In C. elegans, the germline is comprised of two main compartments, a mitotic region (the premeiotic tip), where nuclei are under the influence of the distal tip cell that maintains them in a proliferative state; and the meiotic region, where nuclei progress through the stages of Prophase I before being fertilized. The mitotic compartment of the worm gonad following BPA exposure was examined. As revealed by DAPI staining, BPA exposed worms showed a significant decrease in the size of the mitotic zone compared to vehicle. Both the total number of mitotic nuclei and the number of rows of nuclei were reduced by half (p=0.01, n=5 gonads) (FIG. 2B). This observation was further supported by immunostaining of worms with the mitotic marker phospho-histone H3. BPA exposed worms displayed a three-fold reduction of pH3 positive nuclei compared to control (p=10⁻⁶, n=10 gonads). To address whether the shorter mitotic region could be due to a general delay in the development of the worm gonad, the size of the premeiotic tip a day later (5 days after the start of exposure) in BPA exposed animals (FIG. 6) was also examined. No significant increase was observed suggesting that the effect of BPA on the mitotic compartment is not due to a general delay in the development of the gonad but rather likely reflects a discrete action of BPA on the worm gonad.

### Perturbation of the Synaptonemal Complex and Chromatin Integrity During Pachytene

Exposure of mice to BPA causes defects in homologous chromosome synapsis in pachytene as revealed by immunostaining for SCP-3, a lateral element component of the synaptonemal complex (SC) [43], leading to the precocious separation of homologous chromosomes at that stage [14]. Additionally, chromatin integrity was affected as revealed by the presence of end-to-end chromosome associations. These abnormalities likely resulted in the increased chromosome nondisjunction highlighted by the greater number of aneuploid eggs and embryos observed [14]. To examine meiotic chromosome synapsis in C. elegans following BPA exposure, assembly of the synaptonemal complex was examined by assessing the localization of the axial element protein HTP-3 (a paralogue of the axial component HIM-3 required for proper synapsis and pairing of homologous chromosomes [44]) and the central region protein SYP-1 [45], as the nuclei enter meiosis. As nuclei exit mitosis and progress into meiosis in wild type germlines, HTP-3 and SYP-1 are assembled onto the chromatin as foci and short tracks which increase in length through the transition zone. By pachytene, the polymerization of the SC is completed and both proteins form fully elongated tracks spanning the entire length of the homologous chromosomes. Following exposure to BPA, the kinetics and extent of assembly of HTP-3 and SYP-1 onto the chromatin during transition zone were not affected when compared to control (n=10 gonads).

In contrast, analysis of pachytene nuclei in BPA treated worms revealed defects in chromosome synapsis as evidenced by the presence of DAPI-stained DNA tracks were devoid of SYP-1 staining. These abnormal nuclei were not simply apoptotic nuclei as they were not specifically found near the bend of the gonad, where germline nuclei were cleared by apoptosis but instead could be found throughout pachytene. As disruption of the integrity of the SC could lead to a separation of the homologous chromosomes, the localization of the X specific pairing center component HIM-8 was examined. In C. elegans, specific loci, termed pairing centers, on chromosomes allow for the physical interaction between homologous pairs. HIM-8 along with other pairing center components associate with the pairing centers and the nuclear envelope and mediate homologous pairing [46, 47]. In control as well as in BPA treated worms, only 1 focus per nucleus throughout pachytene (n=7 gonads) was observed, indicating that although BPA alters SYP-1 distribution, it does not lead to unpairing of homologous chromosomes. Interestingly, however, the presence of DAPI stained bodies or fragments was observed in about 10% of pachytene nuclei (n=84). In some instances, these chromatin regions were still connected to chromosomes as suggested by the appearance of large foci of RAD-51, a protein involved in strand invasion/exchange during double-strand break repair [48]; specifically at the connection between the rest of the nucleus and the DAPI stained body. Together, these results indicate that following exposure to BPA, while the kinetics of establishment of synapsis appears normal, chromatin and SC integrity are impaired.

### Altered DNA Repair Progression in the Germline and Checkpoint Activation

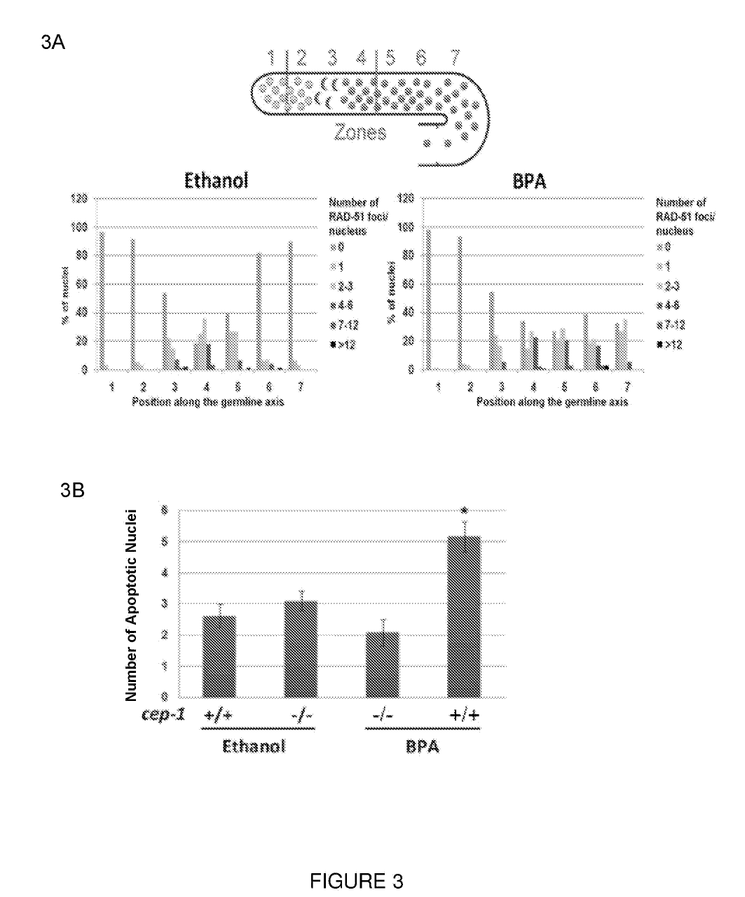

Synapsis and recombination are tightly linked events during pachytene throughout various species, as it is only in the context of a fully assembled SC that crossover recombination can be completed. Indeed, in C. elegans, null mutants of SC components such as syp-1, syp-2, syp-3, syp-4 and him-3, increased levels of RAD-51 foci formation and delayed kinetics of foci turnover are observed during pachytene. Consequently, there is activation of the DNA damage checkpoint leading to increased germ cell apoptosis [25, 45, 49, 50]. Recombination is an essential and highly regulated process for the exchange of information that takes place during prophase I [18]. As nuclei exit mitosis and progress through meiosis, programmed meiotic DNA double strand breaks (DSBs) are formed and processed by several DNA repair proteins including RAD-51 [25, 48]. RAD-51 levels vary greatly during progression through prophase I as it is loaded onto DNA during the process of strand invasion and is removed as DSBs repair progresses. Therefore, RAD-51 foci are rare during mitosis, found at moderate to high levels during early to mid-pachytene and are dramatically decreased in numbers by late pachytene (see diagram in FIG. 3A) [25, 48]. Indeed, in ethanol exposed worms, RAD-51 levels follow these kinetics and RAD-51 foci are reduced to minimal levels by late pachytene (zones 6 and 7) (FIG. 3A). In contrast, worms exposed to BPA still display elevated number of RAD-51 foci in these zones indicating that BPA exposure alters the progression of DSB repair during meiosis. As unrepaired recombination intermediates can trigger a DNA-damage checkpoint during pachytene leading to increased germ cell apoptosis [51], the number of apoptotic nuclei following BPA exposure was measured by Syto-12 staining [52]. Gonads treated with BPA displayed a two to three-fold increase in number of apoptotic nuclei compared to vehicle (p=0.0001, n=12). The C. elegans p53 homologue CEP-1 is required for activation of apoptosis in response to DNA damage. The elevated apoptotic levels observed in response to BPA exposure were reduced to normal levels in a cep-1 mutant background (p=0.35, n=12) indicating that BPA induction of apoptosis is cep-1-dependent (FIG. 3A).

ATL-1, the C. elegans homologue of mammalian ATM and Rad3-related protein (ATR), accumulates to sites of persistent DSBs during meiosis [53], and together with CLK-2, acts as a sensor of germ-cell DNA damage which leads to phosphorylation of the checkpoint kinase CHK1 and ultimately apoptosis [53-55]. To further examine the progression of DSB repair, the levels of ATL-1 and phosphorylated CHK1 (p-CHK1) were monitored throughout the gonad following exposure to BPA. Consistent with the persistence of RAD-51 foci and the activation of the DNA damage checkpoint, a significant increase in the levels of ATL-1 and p-CHK1 signals on BPA treated pachytene nuclei, compared to control, was observed (n=11 and n=7 respectively).

Notably, elevated RAD-51, ATL-1 or p-CHK1 staining in the mitotic germline nuclei was not observed, indicating that BPA exposure elicits a specific stress on the progression of meiotic DNA repair that ultimately leads to activation of the DNA damage checkpoint and clearing by apoptosis of the most affected nuclei.

### BPA Causes Severe Defects at Diakinesis

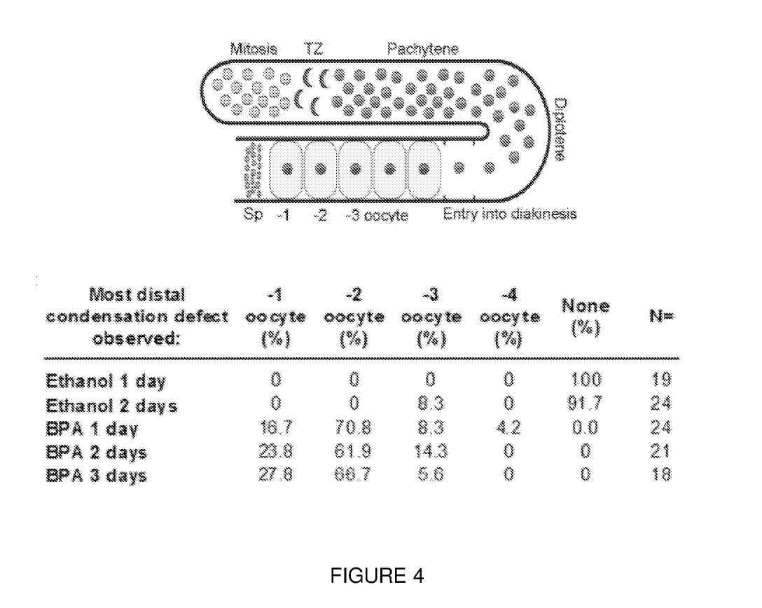

Proper processing of recombination events is coupled with the formation of mature bivalents during the diakinesis stage such that in mutants where recombination intermediate processing is impaired, bivalent maturation is delayed [27, 50]. To assess whether the genomic and SC integrity impairment observed at pachytene correlated with defects later during prophase I, the morphology of diakinesis nuclei following BPA or vehicle treatment was examined. In ethanol treated control gonads, normal kinetics of bivalent maturation was observed, culminating in the formation of 6 highly condensed DAPI stained bodies representing the 6 pairs of homologous chromosomes held together by chiasmata, the physical result of crossing overs. In contrast, bivalents in diakinesis oocytes showed impaired condensation in BPA treated gonads. Specifically, in 23% of all gonads analyzed (n=63), the oocyte most proximal to the spermatheca, termed the −1 oocyte, did not show normal chromosome condensation compared to control (FIG. 4). Furthermore, in all BPA gonads examined, some level of delay in bivalent maturation as judged by nuclear morphology was observed manifesting itself either starting from the −1, −2 or −3 oocyte (FIG. 4). This aberrant kinetics of bivalent formation was not caused by a general delay in maturation of the gonad as the worms collected for analysis both formed and laid eggs and also did not show an improvement in bivalent maturation in subsequent days after initial analysis (17% of −1 oocytes show lack of condensation one day post-L4 compared to 24 and 28% two and three days post-L4, n=24, 21, and 18 respectively).

Associated with the changes in chromatin morphology, the SC is disassembled during late diakinesis in a tightly controlled fashion. Accordingly, SC components, such as SYP-1, are detectable on the short arms of the bivalent in the −3 oocyte but are essentially absent from the bivalents of −2 and −1 oocytes [50, 56]. Thus, whether a delay in chromosome remodeling could correlate with an incomplete disassembly of SC components in late diakinesis was examined. Indeed, while only 7% (n=16) of ethanol treated gonads displayed faint SYP-1 signal on the short arm of the bivalent in the −2 oocyte, BPA treated gonads showed residual staining in 80% of all gonads. Furthermore, 10% of BPA exposed gonads also showed SYP-1 staining on the short arm of the bivalent in the −1 oocyte which was never observed in the ethanol control group (n=15).

Accurate chromosome segregation at meiosis I is characterized by the carefully orchestrated loss of cohesion between homologs coupled with retention of cohesion between sister chromatids. This regulated process involves the targeting of the Aurora-B kinase AIR-2, a member of the chromosomal passenger complex, to the short arm of the bivalents in the −1 oocyte [56-58] which has been proposed to lead to the removal the meiosis-specific cohesin REC-8 from the short arms of the bivalent [57, 58]. To examine whether these key steps in late meiotic prophase I might be impaired in BPA treated gonads, the localization of AIR-2 in the −1 oocytes was examined. AIR-2 was not detected on the bivalents of 100% of the gonads treated with BPA as compared to 10% of ethanol treated controls (n=15). Phospho histone H3 (pH3) is a canonical substrate for the activity of AIR-2 [59] and accordingly, although it accumulates in the −3 to −1 oocytes, it is only detected on the short arms of the bivalent in the −1 oocyte in the presence of AIR-2 [56, 57]. BPA treated oocytes showed a delay in the kinetics of pH3 nuclear accumulation with only the −1 oocyte showing detectable levels of pH3. Furthermore, although pH3 did accumulate in the nucleus, pH3 staining was only localized to the bivalents in 14% of all BPA treated −1 oocytes (n=7). Taken together, these results indicate that the timing of bivalent maturation is delayed following BPA exposure compared to ethanol control.

### Chromosomal Aberrations are Apparent Following Fertilization

Considering the extent of the defects observed before fertilization, during late diakinesis, it was predicted that the early steps of embryogenesis, notably the first cell division would be impaired. 60 first mitosis in ethanol and BPA exposed H2B::mCherry; γ-tubulin::GFP transgenic worms were captured by live imaging. While normal pronuclear fusion and chromosomal alignment and segregation in ethanol controls was observed, the first division of BPA exposed embryos was highly abnormal. DNA masses along with individual chromosomes were apparent in one-cell embryos which ultimately failed to align on the metaphase plate and/or segregate properly (n=9/60). In some instances, multiple spindle poles were apparent. These embryos failed to undergo cytokinesis and progress to the 2-cell stage.

### BPA Exerts Anti-Estrogenic Effects on the C. Elegans Germline

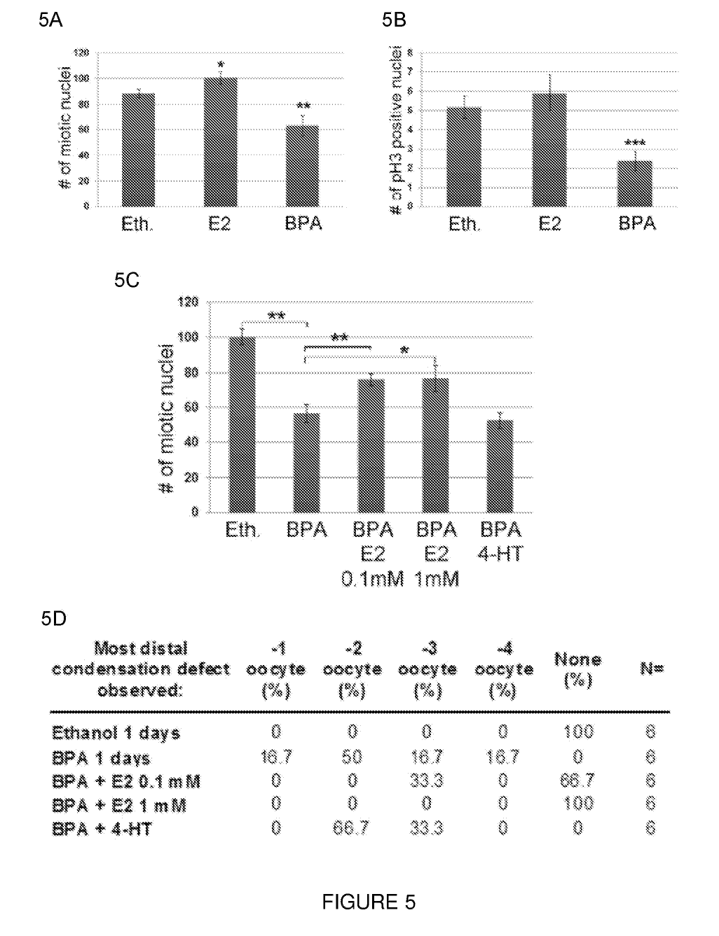

BPA bears endocrine disrupting activity in many developmental contexts including gonadal, brain and mammary gland development [60-63]. BPA has affinity for multiple members of the estrogen receptors family, in particular BPA binds to the human estrogen receptor alpha (ERα) and ERβ as well as estrogen-related receptor gamma (ERR-γ) [64-66] and in C. elegans with nuclear hormone receptor NHR-14 which also binds estrogen [67]. Surprisingly, in the context of oogenesis, evidence suggests that BPA acts as an estrogen antagonist as the defects observed at the pachytene stage in BPA treated animal also occurred at comparable frequencies in ERβ−/− mice. Furthermore, BPA exposure did not lead to increased chromosomal aberrations in these knock-out mice [14]. To investigate whether BPA action on the C. elegans gonad is also mediated by its estrogen modulator activity, the effect of BPA was compared with the effect of estradiol (E2), a canonical estrogen, as well as 4-hydroxy-tamoxifen (4-HT), an estrogen modulator. Two hallmarks of exposure to BPA were examined, the decrease in the size of the proliferative zone and the condensation defects observed at diakinesis, as they are both highly penetrant and consistent phenotypes. Compared to ethanol control gonads, worms exposed to 1 mM E2 for 4 days showed a modest but significant 13% increase in the number of nuclei comprising the mitotic region as judged by DAPI staining (FIG. 5A, p=0.05; n=11). Exposure of worms to 0.15 mM 4-HT (maximum concentration dissolvable in medium) however led to a marked 30% decrease of the mitotic zone (p=0.01, n=7). This result was confirmed by immunostaining for the mitotic marker pH3, 4-HT gonads showed a two-fold decrease in pH3 positive nuclei in their mitotic zone compared to ethanol alone (FIG. 5B, p=0.001, n=14). The effects of 4-HT on the mitotic zone were correlated with condensation defects at diakinesis although these defects were not as severe as when exposed to BPA. Indeed, while 1 mM E2 exposure did not cause any observable bivalent maturation defects at diakinesis, 0.15 mM 4-HT treatment led to moderate condensation defects including the presence of chromatin bridges in the −1 oocyte. The opposing effects of E2 and 4-HT and the similarity of action of 4-HT and BPA on the mitotic zone and bivalent formation suggest that 4-HT and BPA may exert their effect on the germline through anti-estrogenic activity. To test this hypothesis, worms were exposed to ethanol, BPA, and either a combination of BPA and E2 (0.1 mM and 1 mM) or BPA and 4-HT. Similarly to the experiments described above, the size of the mitotic zone was quantified as well as condensation defects at diakinesis. As expected, BPA decreases the size of the mitotic zone by half (FIG. 5C). Co-exposure of BPA with 4-HT did not significantly decrease the size of the mitotic zone further. Treatment with both BPA and E2 at either concentration however significantly increased the size of the mitotic zone (p=0.01, 0.1 mM, and p=0.05, 1 mM, n=5, respectively). Similarly, co-exposure of BPA with E2 at either concentration rescued the diakinesis condensation defects (FIG. 5D). Thus, these results strongly imply that the action of BPA on the mitotic zone and in diakinesis are caused by BPA hormonal activity and more specifically, by its anti-estrogenic activity.

### Methods of the Invention

Growing Conditions and Drug Exposures. Worms were cultured according to [23] at 20° C. on NGM plates without cholesterol. Bisphenol-A, estradiol and 4-hydroxy-tamoxifen were purchased from Sigma-Aldrich, dissolved in 100% ethanol and added to the hot plate medium prior to pouring for a final ethanol concentration of 0.1%. Exposure was carried from embryogenesis to adulthood by plating sodium hypochloride treated eggs [24] onto drug and control plates and incubated at 20° C. for four days.

Rad-51 Time Course Analysis. The number of RAD-51 foci and their kinetics throughout the seven zones was assessed as described in [25]. On average ethanol exposed worms had the following number of nuclei per zone: zone 1 (n=46), zone 2 (n=55), zone 3 (n=54), zone 4 (n=57), zone 5 (n=54), zone 6 (n=47), zone 7 (n=40). BPA exposed worms nuclei per zones were: zone 1 (n=44), zone 2 (n=40), zone 3 (n=41), zone 4 (n=34), zone 5 (n=27), zone 6 (n=28), zone 7 (n=21).

Quantification of Germ Cell Apoptosis. Apoptotic germline nuclei were scored in young adults between 20 and 24 hours post-L4 using SYTO-12 (Invitrogen) and were processed as described in [26]. The balanced cep-1 mutant strain genotype is cep-1 (lg12501)I/hT2 (gIs48) I;III. The strain was maintained as GFP positive balanced heterozygotes. Both GFP positive and negative worms (heterozygous and homozygous for cep-1 respectively) were analyzed following BPA exposure. Statistical analysis was performed by using a student paired t-test.

Antibody Staining. DAN staining, immunostaining and analysis of stained meiotic nuclei were performed as in [25] except for ATL-1 and AIR-2 antibodies were dissected gonads were subjected to a 1% fixation method as described in [27]. Antibodies were used at the following dilutions: rabbit α-SYP-1 (1:100), mouse α-RAD-51 (1:100), rabbit α-HTP-3 (1:500), guinea pig α-HIM-8 (1:100), rabbit α-ATL-1 (1:50), rabbit α-AIR-2 (1:100) and rabbit α-pCHK-1 (1:100). The secondary antibodies used were: Cy3 anti-rabbit, FITC anti-guinea-pig and FITC anti-mouse (Jackson Immunochemicals), each at 1:100, Stacks of 0.2 μm slices were captured using a Leica microscope, coupled to the DeltaVision fluorescence microscope system (Applied Precision). The images were projections approximately halfway through 3D data stacks of whole nuclei, except for diakinesis images which encompass entire nuclei.

Live Imaging. For live imaging, a pie-1::mCherry::H2B; pie-1::GFP::gamma tubulin OD57 strain was used [28]. The strain was incubated at 25° C. 24 hours prior to imaging. Images were captured using the Leica-Deltavision system described above. Worms were immobilized on an agarose pad by sectioning of the head and images of the first cell division were captured at a rate of one image every 10 seconds for 5 minutes.

### REFERENCES FOR EXAMPLE 1

1. Hunt, P. A. and T. J. Hassold, Human female meiosis: what makes a good egg go bad? Trends Genet, 2008. 24(2): p. 86-93.
2. Christianson, R. E., S. L. Sherman, and C. P. Torfs, Maternal meiosis II nondisjunction in trisomy 21 is associated with maternal low socioeconomic status. Genet Med, 2004. 6(6): p. 487-94.
3. Hunt, P. A., Meiosis in mammals: recombination, non-disjunction and the environment. Biochem Soc Trans, 2006. 34(Pt 4): p. 574-7.
4. Collins, F. S., G. M. Gray, and J. R. Bucher, Toxicology. Transforming environmental health protection. Science, 2008. 319(5865): p. 906-7.
5. Geens, T., et al., Assessment of human exposure to Bisphenol-A, Triclosan and Tetrabromobisphenol-A through indoor dust intake in Belgium. Chemosphere, 2009. 76(6): p. 755-60.
6. Chapin, R. E., et al., NTP-CERHR expert panel report on the reproductive and developmental toxicity of bisphenol A. Birth Defects Res B Dev Reprod Toxicol, 2008. 83(3): p. 157-395.
7. NTP-CERHR Monograph on the Potential Human Reproductive and Developmental Effects of Bisphenol A. NTP CERHR MON, 2008 (22): p. i-III1.
8. Calafat, A. M., et al., Urinary concentrations of bisphenol A and 4-nonylphenol in a human reference population. Environ Health Perspect, 2005. 113(4): p. 391-5.
9. Lang, I. A., et al., Association of urinary bisphenol A concentration with medical disorders and laboratory abnormalities in adults. JAMA, 2008. 300(11): p. 1303-10.
10. Li, D., et al., Occupational exposure to bisphenol-A (BPA) and the risk of self-reported male sexual dysfunction. Hum Reprod, 2010. 25(2): p. 519-27.
11. Maffini, M. V., et al., Endocrine disruptors and reproductive health: the case of bisphenol-A. Mol Cell Endocrinol, 2006. 254-255: p. 179-86.
12. Palanza, P., et al., Effects of developmental exposure to bisphenol A on brain and behavior in mice. Environ Res, 2008. 108(2): p. 150-7.
13. Hunt, P. A., et al., Bisphenol a exposure causes meiotic aneuploidy in the female mouse. Curr Biol, 2003. 13(7): p. 546-53.
14. Susiarjo, M., et al., Bisphenol A exposure in utero disrupts early oogenesis in the mouse. PLoS Genet, 2007. 3(1): p. e5.
15. Leung, M. C., et al., Caenorhabditis elegans: an emerging model in biomedical and environmental toxicology. Toxicol Sci, 2008. 106(1): p. 5-28.
16. Kaletta, T. and M. O. Hengartner, Finding function in novel targets: C. elegans as a model organism. Nat Rev Drug Discov, 2006. 5(5): p. 387-98.
17. Colaiacovo, M. P., The many facets of SC function during C. elegans meiosis. Chromosoma, 2006. 115(3): p. 195-211.
18. Garcia-Muse, T. and S. J. Boulton, Meiotic recombination in Caenorhabditis elegans. Chromosome Res, 2007. 15(5): p. 607-21.
19. Martinez-Perez, E. and M. P. Colaiacovo, Distribution of meiotic recombination events: talking to your neighbors. Curr Opin Genet Dev, 2009. 19(2): p. 105-12.
20. Pawlowski, W. P. and W. Z. Cande, Coordinating the events of the meiotic prophase. Trends Cell Biol, 2005. 15(12): p. 674-81.
21. Zetka, M., Homologue pairing, recombination and segregation in Caenorhabditis elegans. Genome Dyn, 2009. 5: p. 43-55.
22. Zetka, M. and A. Rose, The genetics of meiosis in Caenorhabditis elegans. Trends Genet, 1995. 11(1): p. 27-31.
23. Brenner, S., The genetics of Caenorhabditis elegans. Genetics, 1974. 77(1): p. 71-94.
24. Stiernagle, T., Maintenance of C. elegans. WormBook, 2006: p. 1-11.
25. Colaiacovo, M. P., et al., Synaptonemal complex assembly in C. elegans is dispensable for loading strand-exchange proteins but critical for proper completion of recombination. Dev Cell, 2003. 5(3): p. 463-74.
26. Gumienny, T. L., et al., Genetic control of programmed cell death in the Caenorhabditis elegans hermaphrodite germline. Development, 1999. 126(5): p. 1011-22.
27. Saito, T. T., et al., Caenorhabditis elegans HIM-18/SLX-4 interacts with SLX-1 and XPF-1 and maintains genomic integrity in the germline by processing recombination intermediates. PLoS Genet, 2009. 5(11): p. e1000735.
28. McNally, K., et al., Katanin controls mitotic and meiotic spindle length. J Cell Biol, 2006. 175(6): p. 881-91.
29. Meeker, J. D., A. M. Calafat, and R. Hauser, Urinary Bisphenol A Concentrations in Relation to Serum Thyroid and Reproductive Hormone Levels in Men from an Infertility Clinic. Environ Sci Technol, 2009.
30. Akingbemi, B. T., et al., Inhibition of testicular steroidogenesis by the xenoestrogen bisphenol A is associated with reduced pituitary luteinizing hormone secretion and decreased steroidogenic enzyme gene expression in rat Leydig cells. Endocrinology, 2004. 145(2): p. 592-603.
31. Savabieasfahani, M., et al., Developmental programming: differential effects of prenatal exposure to bisphenol-A or methoxychlor on reproductive function. Endocrinology, 2006. 147(12): p. 5956-66.
32. Salian, S., T. Doshi, and G. Vanage, Neonatal exposure of male rats to Bisphenol A impairs fertility and expression of sertoli cell junctional proteins in the testis. Toxicology, 2009. 265(1-2): p. 56-67.
33. Lahnsteiner, F., et al., Effect of bisphenol A on maturation and quality of semen and eggs in the brown trout, Salmo trutta f. fario. Aquat Toxicol, 2005. 75(3): p. 213-24.
34. Kato, H., et al., Effects of bisphenol A given neonatally on reproductive functions of male rats. Reprod Toxicol, 2006. 22(1): p. 20-9.
35. Mok-Lin, E., et al., Urinary bisphenol A concentrations and ovarian response among women undergoing IVF. Int J Androl, 2009.
36. Sugiura-Ogasawara, M., et al., Exposure to bisphenol A is associated with recurrent miscarriage. Hum Reprod, 2005. 20(8): p. 2325-9.
37. Watanabe, M., et al., A mutation in a cuticle collagen causes hypersensitivity to the endocrine disrupting chemical, bisphenol A, in Caenorhabditis elegans. Mutat Res, 2005. 570(1): p. 71-80.
38. Kabuto, H., et al., Effects of bisphenol A on the metabolisms of active oxygen species in mouse tissues. Environ Res, 2003. 93(1): p. 31-5.
39. Moors, S., P. Diel, and G. H. Degen, Toxicokinetics of bisphenol A in pregnant DA/Han rats after single i.v. application. Arch Toxicol, 2006. 80(10): p. 647-55.
40. Vandenberg, L. N., et al., Human exposure to bisphenol A (BPA). Reprod Toxicol, 2007. 24(2): p. 139-77.
41. He, Y., et al., Occupational exposure levels of bisphenol A among Chinese workers. J Occup Health, 2009. 51(5): p. 432-6.
42. Schonfelder, G., et al., Parent bisphenol A accumulation in the human maternal-fetal-placental unit. Environ Health Perspect, 2002. 110(11): p. A703-7.
43. Yuan, L., et al., The murine SCP3 gene is required for synaptonemal complex assembly, chromosome synapsis, and male fertility. Mol Cell, 2000. 5(1): p. 73-83.
44. Goodyer, W., et al., HTP-3 links DSB formation with homolog pairing and crossing over during C. elegans meiosis. Dev Cell, 2008. 14(2): p. 263-74.
45. MacQueen, A. J., et al., Synapsis-dependent and -independent mechanisms stabilize homolog pairing during meiotic prophase in C. elegans. Genes Dev, 2002. 16(18): p. 2428-42.
46. Phillips, C. M., et al., HIM-8 binds to the X chromosome pairing center and mediates chromosome-specific meiotic synapsis. Cell, 2005. 123(6): p. 1051-63.
47. Phillips, C. M. and A. F. Dernburg, A family of zinc finger proteins is required for chromosome-specific pairing and synapsis during meiosis in C. elegans. Dev Cell, 2006. 11(6): p. 817-29.
48. Rinaldo, C., et al., Roles for Caenorhabditis elegans rad-51 in meiosis and in resistance to ionizing radiation during development. Genetics, 2002. 160(2): p. 471-9.
49. Smolikov, S., K. Schild-Prufert, and M. P. Colaiacovo, A yeast two-hybrid screen for SYP-3 interactors identifies SYP-4, a component required for synaptonemal complex assembly and chiasma formation in Caenorhabditis elegans meiosis. PLoS Genet, 2009. 5(10): p. e1000669.
50. Nabeshima, K., A. M. Villeneuve, and M. P. Colaiacovo, Crossing over is coupled to late meiotic prophase bivalent differentiation through asymmetric disassembly of the SC. J Cell Biol, 2005. 168(5): p. 683-9.
51. Gartner, A., et al., A conserved checkpoint pathway mediates DNA damage—induced apoptosis and cell cycle arrest in C. elegans. Mol Cell, 2000. 5(3): p. 435-43.
52. Gartner, A., P. R. Boag, and T. K. Blackwell, Germline survival and apoptosis. WormBook, 2008: p. 1-20.
53. Garcia-Muse, T. and S. J. Boulton, Distinct modes of ATR activation after replication stress and DNA double-strand breaks in Caenorhabditis elegans. EMBO J, 2005. 24(24): p. 4345-55.
54. Moser, S. C., et al., Functional dissection of Caenorhabditis elegans CLK-2/TEL2 cell cycle defects during embryogenesis and germline development. PLoS Genet, 2009. 5(4): p. e1000451.
55. Zhao, H. and H. Piwnica-Worms, ATR-mediated checkpoint pathways regulate phosphorylation and activation of human Chk1. Mol Cell Biol, 2001. 21(13): p. 4129-39.
56. de Carvalho, C. E., et al., LAB-1 antagonizes the Aurora B kinase in C. elegans. Genes Dev, 2008. 22(20): p. 2869-85.
57. Kaitna, S., et al., The aurora B kinase AIR-2 regulates kinetochores during mitosis and is required for separation of homologous Chromosomes during meiosis. Curr Biol, 2002. 12(10): p. 798-812.
58. Rogers, E., et al., The aurora kinase AIR-2 functions in the release of chromosome cohesion in Caenorhabditis elegans meiosis. J Cell Biol, 2002. 157(2): p. 219-29.
59. Giet, R. and D. M. Glover, Drosophila aurora B kinase is required for histone H3 phosphorylation and condensin recruitment during chromosome condensation and to organize the central spindle during cytokinesis. J Cell Biol, 2001. 152(4): p. 669-82.
60. Rubin, B. S., et al., Evidence of altered brain sexual differentiation in mice exposed perinatally to low, environmentally relevant levels of bisphenol A. Endocrinology, 2006. 147(8): p. 3681-91.
61. Rubin, B. S., et al., Perinatal exposure to low doses of bisphenol A affects body weight, patterns of estrous cyclicity, and plasma LH levels. Environ Health Perspect, 2001. 109(7): p. 675-80.
62. Vandenberg, L. N., et al., Perinatal exposure to the xenoestrogen bisphenol-A induces mammary intraductal hyperplasias in adult CD-1 mice. Reprod Toxicol, 2008. 26(3-4): p. 210-9.
63. Welshons, W. V., S. C. Nagel, and F. S. vom Saal, Large effects from small exposures. III. Endocrine mechanisms mediating effects of bisphenol A at levels of human exposure. Endocrinology, 2006. 147(6 Suppl): p. S56-69.
64. Matsushima, A., et al., Structural evidence for endocrine disruptor bisphenol A binding to human nuclear receptor ERR gamma. J Biochem, 2007. 142(4): p. 517-24.
65. Kuiper, G. G., et al., Interaction of estrogenic chemicals and phytoestrogens with estrogen receptor beta. Endocrinology, 1998. 139(10): p. 4252-63.
66. Gould, J. C., et al., Bisphenol A interacts with the estrogen receptor alpha in a distinct manner from estradiol. Mol Cell Endocrinol, 1998. 142(1-2): p. 203-14.
67. Mimoto, A., et al., Identification of an estrogenic hormone receptor in Caenorhabditis elegans. Biochem Biophys Res Commun, 2007. 364(4): p. 883-8.

### Example 2

### High-Throughput Screening of Environmental Factors for Reproductive Toxicity and Fat Homeostasis Disruption

The following describes the use of the genetic model organism C. elegans, a commonly used laboratory organism, as a model for investigating the influence of environmental factors on reproduction (specifically meiosis) and also fat homeostasis.

Several genetic, cytological, molecular and biochemical technologies were employed in C. elegans for the screening of environmental compounds to identify reproductive toxicants. There is currently no screening tool that allows the determination of a chemical as affecting these processes. These strategies are fast, reliable, inexpensive and can be easily scaled up to accommodate the need for high volume, large sale screening. The research described herein can greatly facilitate the identification of chemicals as meiotic disruptors in toxicological screenings. Furthermore, the nematode C. elegans can be used in the context of high-throughput screening of environmental compounds for their biological effects.

### A Screening Strategy for the Identification of Compounds Disrupting Meiosis Using C. Elegans

Despite the devastating outcomes of genetic anomalies stemming from problems during the cell division program known as meiosis, the screening of environmental toxicants for their ability to disrupt the process of meiosis and the dissection of affected molecular pathway has revealed particularly challenging. Meiosis is a complex process which cannot be recapitulated in a tissue culture setting. Mammalian female meiosis from inception to completion spans from several months in mice to several decades in humans. It is initiated early on during embryogenesis, where key events guiding chromosomal segregation and exchange take place, and is only completed at puberty during ovulation. Each of these crucial meiotic steps, namely chromosome pairing, synapsis and recombination, all part of meiotic prophase I, occur at specific stages of development within the confines of the embryonic gonad and are therefore not easily accessible for study. Due to time, cost, experimental and ethical constraints, the study of environmental effect on the process of meiosis is therefore very challenging. Furthermore, there is currently no high-throughput alternative approach for the screening of environmental disruption of meiosis.

For this purpose, the nematode Caenorhabditis elegans, a commonly used genetic model, offers multiple advantages that fit well within the current reshaping of modern toxicology (discussed in Collins, Gray and Bucher, 2008). Due to its small size, low cost, short reproduction cycle and ease of manipulation, it is extremely amenable not only to the detailed analysis of affected biological processes, but also to large scale, high-throughput screenings. Indeed worms can be grown either on 96-well plates or in high volume liquid cultures. As they are transparent, microscopy and detection of endogenous fluorescent reporters can be easily performed. C. elegans constitutes an ideal model for the study of meiosis as the worm gonad comprises almost 50% of the total number of nuclei in the adult worm. Moreover, C. elegans meiosis progresses both in a temporal and spatial gradient such that nuclei in each phase of meiosis are found in a specific region of the gonad making their identification and analysis straightforward. Most importantly, C. elegans is a relevant model system for the study of meiosis as many cellular and molecular pathways are evolutionarily conserved between the worm and mammals. Indeed, genetic screens performed in the nematode have led to a dramatic increase in our understanding of the genes involved in the regulation of meiosis in C. elegans as well as in mammals (reviewed in Pawlowski and Cande, 2005; Colaiacovo, 2006). Using the remarkable features of C. elegans biology, a powerful new strategy to address the need for the fast screening and characterization of environmental meiotic disruptors has been developed.

The screening strategy incorporates and adapts a screening strategy first developed in C. elegans for the isolation of meiotic mutants. This method has been adapted to create an inexpensive and reliable, high-throughput screening tool for the identification and subsequent characterization of the effect of individual compounds or mixture of compounds on meiosis. Prior to the results reported herein, it was not known whether or not such a system could be used to identify environmental agents that lead to meiotic disruption in C. elegans. Furthermore, prior to the results reported above in Example 1, it was not known whether any such agents identified in the C. elegans system would have physiological relevance to mammalian systems. It is now known that the results of such assays and of the further characterization on the meiotic effects, have direct application to mammalian systems.

The screening strategy was initially designed for the identification of meiotic mutants (Kelly et al, 2000). This approach, termed the “Green eggs and Him” screen, takes advantage of the rare proportion of male (XO) progeny (<0.2%) laid by wild type hermaphroditic (XX) worms (Hodgkin et al., 1979). These males naturally arise in the population as a result of non-disjunction and therefore improper segregation of the X chromosomes during meiosis. Consequently, as disruption of meiotic progression very frequently leads to increased non-disjunction, errors in meiosis correlate with an elevated fraction of males, or a High Incidence of Males (Him), in the worm population. To circumvent possible embryonic lethality and for ease of screening, Kelly and colleagues also added to their screening strategy a fluorescent reporter that allows for rapid identification of males. Indeed, by fusing the promoter sequence of a male specific gene (xol-1) expressed very early during embryogenesis to the Green Fluorescent Protein (GFP) coding sequence, they were able to quickly identify male embryos within the adult worm's uterus by simple fluorescent microscopy of live worms. The use of the xol-1::GFP reporter strain and selection of GFP positive worms in their screen led to the identification of a key meiotic component involved in the DNA repair pathway (Kelly et al, 2000). That approach has been adapted in two different ways to the screening of compounds for their ability to disrupt meiosis.

### GFP Based Assay

Worms are grown from eggs to adulthood (4 days) on 96-well plates containing media mixed with the compound of interest. Initially, two different concentrations are assayed, 1 μM and 100 μM, concentrations previously used in the context of other C. elegans toxicological screens (Boyd, McBride and Freedman, 2007; Boyd et al, 2009).

Following exposure, the worms are washed off the plate with buffer and run through a high-throughput flow cytometry device, commonly known as a “worm sorter” (COPAS-Biosort from Union Biometrica). The worm sorter not only directs the sorting of GFP positive worms from GFP negative ones but when coupled to the Profiler analysis software, also allows for the localization of the fluorescence within the worm. Scoring in the screen, is for GFP positive eggs present in the animal's mid-ventral section (uterus), therefore, any worms showing mis-localized GFP expression are discounted thus improving the accuracy of the screen.

In preliminary experiments, the COPAS-Biosort and the Profiler software were used to examine and compare two strains of worms. The first one carries the xol-1::GFP reporter in the background of a mutation in the him-8 gene that leads to a high incidence of males. In this background, approximately 30% of all embryos are male and express GFP as verified by fluorescence microscopy. The other strain we used only carried the xol-1::GFP reporter. After running these two populations of worms through the worm sorter and profiler, a large population of GFP positive eggs was detected in the xol-1::GFP;him-8 strain and not in the xol-1::GFP strain alone. It was also established that, in this context, the rate of false positives is extremely low. The xol-1::GFP strain can be used to screen environmentally relevant libraries of compounds such as the “1408” library available through the National Toxicology program.

The xol-1::GFP reporter strain was tested for meiotic disruption by addition of an external agent, by exposing the C. elegans to several cell cycle disruptors (microtubule disrupting agents) dissolved in DMSO, DMSO alone was used as a negative control. Each individual test agent was added to the liquid culture medium of the C. elegans at 1 day post-L4 (4 days of development) for a total of 20 hours. The C. elegans was then analyzed for the percentage of male embryos in the adult-midventral sections. Nocodazol was added to the medium at a final concentration of 0.1 mg/ml. This resulted in 37% male embryos (N=16). Demecolcine was added to the liquid culture medium at a final concentration of 0.1 mg/ml resulted in 2.7% male embryos (n=37). DMSO was added to the liquid culture medium at a final concentration of 1%, and used alone resulted in 0% male embryos (n=50).

### Luciferase Based Assay

A variation of the technology described above is also being developed. In this version of the screen, xol-1 driven GFP will be replaced by another reporter gene, in this case the firefly luciferase enzyme. Besides male specific expression of firefly luciferase, the transgenic worms will also carry the Renilla luciferase under the control of a ubiquitous promoter. Both types of luciferases can be assayed independently and simultaneously, Renilla luciferase emission serving as background level and correcting for lethality or slow growth of the worms. This new worm strain, xol-1::LucP; Ub::LucR, just like its GFP equivalent, will be exposed to library of compounds on 96-well plates. After 4 days of exposure, the worms will be exposed to the luciferases substrates and the light emitted will be measured by a luminometer. The compounds giving rise to males and therefore disrupting meiosis, will have a higher than normal ratio of LucP to LucR. Luciferase allows extreme sensitivity (at the attomole level) with very low background activity, as the enzyme is not naturally found in C. elegans. The positive hits from this screen will be validated by a secondary screen using the xol-1::GFP strain. This will allow visual confirmation of the presence of male embryos (green eggs) and will therefore increase the screen's accuracy.

### A Screening Strategy for the Identification of Compounds Affecting Fat Homeostasis

The assays described herein can also greatly facilitate the identification of chemicals as fat homeostasis disruptors in toxicological screenings. A fascinating new avenue in toxicology is the environmental regulation of fat metabolism by compounds termed “obesogens” (Grun and Blumberg, 2006; Grun and Blumberg, 2009). Interestingly, fat metabolism pathways are also conserved between the worm and humans, and C. elegans is increasingly viewed as a powerful model to understand the genetic pathways regulating fat synthesis and adiposity (Schlegel and Stainier, 2007; Watts, 2009). Fat content can easily be detected in C. elegans by staining the worms with various chemicals including oil red O (Kimura et al, 1997; Ashrafi et al, 2003, O'Rourke et al, 2009). In this “fat screen”, worms will be exposed for 4 days to the environmental compounds library and then treated overnight with oil red O for staining of the fat (as described in O'Rourke et al, 2009). The fat will stain red and the intensity of red staining can be captured and analyzed with a regular light microscope coupled to a color camera and computer. Positive hits will be compounds dramatically affecting: (1) the intensity of red staining (more or less fat) and (2) the localization of the red staining (redistribution of fat storage).

### REFERENCES FOR EXAMPLE 2

1. Ashrafi K, Chang F Y, Watts J L, Fraser A G, Kamath R S, Ahringer J, Ruvkun G. Genome-wide RNAi analysis of Caenorhabditis elegans fat regulatory genes. Nature. 2003 Jan. 16; 421(6920):268-72.
2. Boyd W A, McBride S J, Freedman J H. Effects of genetic mutations and chemical exposures on Caenorhabditis elegans feeding: evaluation of a novel, high-throughput screening assay. PLoS One. 2007 Dec. 5; 2(12):e1259.
3. Boyd W A, Smith M V, Kissling G E, Freedman J H. Medium- and high-throughput screening of neurotoxicants using C. elegans. Neurotoxicol Teratol. 2009 Jan. 6.
4. Colaiácovo M P. The many facets of SC function during C. elegans meiosis. Chromosoma. 2006 June; 115(3):195-211. Epub 2006 Mar. 23.
5. Collins F S, Gray G M, Bucher J R. Toxicology. Transforming environmental health protection. Science. 2008 Feb. 15; 319(5865):906-7.
6. Grün F, Blumberg B. Perturbed nuclear receptor signaling by environmental obesogens as emerging factors in the obesity crisis. Rev Endocr Metab Disord. 2007 June; 8(2):161-71.
7. Grun F, Blumberg B. Endocrine disrupters as obesogens. Mol Cell Endo. 2009 May 25; 304(1-2):19-29.
8. Hunt P A, Hassold T J. Human female meiosis: what makes a good egg go bad? Trends Genet. 2008 February; 24(2):86-93.
9. Hodgkin J, Horvitz H R, Brenner S. Nondisjunction Mutants of the Nematode CAENORHABDITIS ELEGANS. Genetics. 1979 January; 91(1):67-94.
10. Kelly K O, Dernburg A F, Stanfield G M, Villeneuve A M. Caenorhabditis elegans msh-5 is required for both normal and radiation-induced meiotic crossing over but not for completion of meiosis. Genetics. 2000 October; 156(2):617-30.
11. Kimura K D, Tissenbaum H A, Liu Y, Ruvkun G. daf-2, an insulin receptor-like gene that regulates longevity and diapause in Caenorhabditis elegans. Science. 1997 Aug. 15; 277(5328):942-6.
12. Pawlowski W P, Cande W Z. Coordinating the events of the meiotic prophase. Trends Cell Biol. 2005 December; 15(12):674-81.
13. O'Rourke E J, Soukas A A, Carr C E, Ruvkun G. C. elegans major fats are stored in vesicles distinct from lysosome-related organelles. Cell Metab. 2009 November; 10(5):430-5.
14. Schlegel A, Stainier D Y. Lessons from “lower” organisms: what worms, flies, and zebrafish can teach us about human energy metabolism. PLoS Genet. 2007 November; 3(11):e199.
15. Susiarjo M, Hassold T J, Freeman E, Hunt P A. Bisphenol A exposure in utero disrupts early oogenesis in the mouse. PLoS Genet. 2007 Jan. 12; 3(1):e5.
16. Watts J L. Fat synthesis and adiposity regulation in Caenorhabditis elegans. Trends Endocrinol Metab. 2009 March; 20(2):58-65. Epub 2009 Jan. 31.

### Example 3

### The Following Pilot Studies were Performed to Further Validate the Methods Described Herein

Two halves of a final screening strain of C. elegans were engineered. A strain carrying the homozygous nx3 cuticle mutation in the context of the xol-1::GFP background was generated, as were several xol-1::luciferase lines. The final strain that will carry both reporters and the cuticle mutation in the homozygous state will be generated by crossing of the two strains.

### Chemical Induction of Aneuploid Embryos

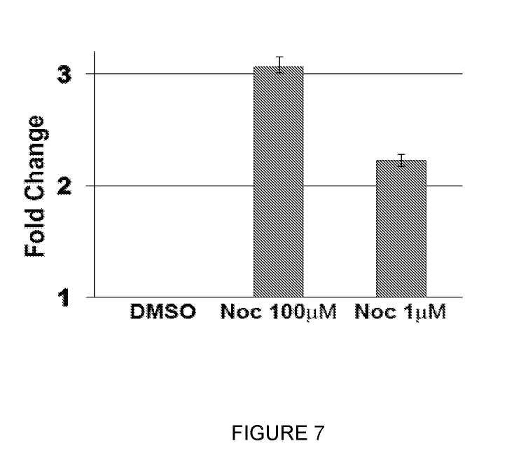

Adult xol-1::GFP worms were exposed to 100 μM nocodazole for 24 hours in liquid culture and the presence of GFP+ embryos in the uterus of exposed worms was monitored. As expected, I detected at least one GFP+ embryo in 55% of all worms compared to 0.3% in control (exposed to solvent DMSO). Similarly, in a preliminary experiment, a significant induction of luciferase was observed following exposure of xol-1::LUC to 1 μM or 100 μM of nocodazole for 24 hours compared to DMSO (FIG. 7).

Testing the Responsiveness (i.e. Chemical Sensitivity) of the System.

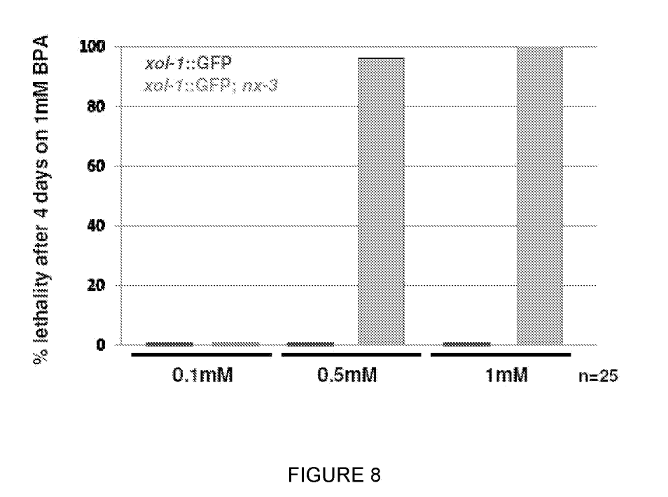

The sensitivity of the xol-1::GFP, nx3 strain compared to the xol-1::GFP strain was assayed by exposing 25 worms for four days (from eggs to adulthood) to a range of concentration of Bisphenol A: 100 μM, 500 μM and 1 mM. The nx3 cuticle mutation was expected to confer increased sensitivity to BPA. Indeed, xol-1::GFP worms showed 0% lethality at all three concentrations tested, while xol-1::GFP, nx3 worms showed 0% lethality at 100 μM, 96% at 500 μM and 100% at 1 Mm (FIG. 8), thus indicating increased sensitivity of the xol-1::GFP, nx3 strain to Bisphenol A.

### Assessing the Relevance of the System by Measuring False Positive and Negative Rates

The effect of selected chemotherapeutic agents on the generation of aneuploidies was analyzed. xol-1::GFP adult worms were exposed to the compounds at a concentration of 100 μM for 24 hours in liquid culture in triplicates. The compounds known to affect chromosome segregation directly such as microtubule inhibitors but also indirectly via induction of DNA damage, which affects meiosis, all showed a significantly elevated number of GFP+ embryos compared to DMSO treated controls. Importantly, chemicals with no previously reported aneugenic activity were not significantly different from control. The results obtained are summarized in the table below:

TABLE 1

| | | Average % of worms |
|---|---|---|
| | | with 1 or more GFP |
| Categories of chemical | Chemicals tested | embryos/category |
| Aneugenic | | |
| Microtubule inhibitor | Nocodazole, Vinblastine | 23.3% * |
| | and Vincristine sulfate | |
| DNA damaging agents | Triethylene melamine, | 3.77% * |
| | Topotecan HCl | |
| Non aneugenic | | |
| Anti-hyperuricemia | Allopurinol | 0.6% |
| MEK inhibitor | 5-iodo | 0.3% |
| EGFR inhibitor | AG1418, Tyr47 | 0.9% |
| Negative control | DMSO | 0.3% |

(* indicates p ≦ 0.05, t-test)

The following NCI chemotherapeutic agents were screened in the above assay.

| | |
|---|---|
| Nocodazole | Microtubule inhibitor |
| Vinblastine | Microtubule inhibitor |
| sulfate | |
| Dactinomycin | Commercial name for Actinomycin D. Inhibits RNA pol |
| | and also replication |
| Vincristine | Microtubule inhibitor |
| sulfate | |
| Bortezomib | Proteasome inhibitor. Worms look very abnormal. |
| | Should DAPI stain |
| TEM | Triethylene melamine. Induces SCE and translocations |
| | in variety of models and systems |
| Methotrexate | Inhibits Dihydrofolate reductase so no folate. So no |
| | Thymidine and no purine. No RNA, DNA or prot |
| | synthesis |
| AG1418 | EGFR inhibitor |
| Tyr47 | EGFR inhibitor |
| DMSO | Negative control |
| Topotecan | Soluble form of Camptothecin. Topoisomerase I |
| HCl | inhibitor. Causes DNA breaks and apoptosis |
| Thioguanine | Inhibits purine synthesis. Induces SSBs. Promotes |
| | cell cycle arrest. |
| 5-iodo | MEK inhibitor |
| Allopurinol | Xanthine oxidase inhibitor. Decreases uric acid and |
| | purine biosynthesis |

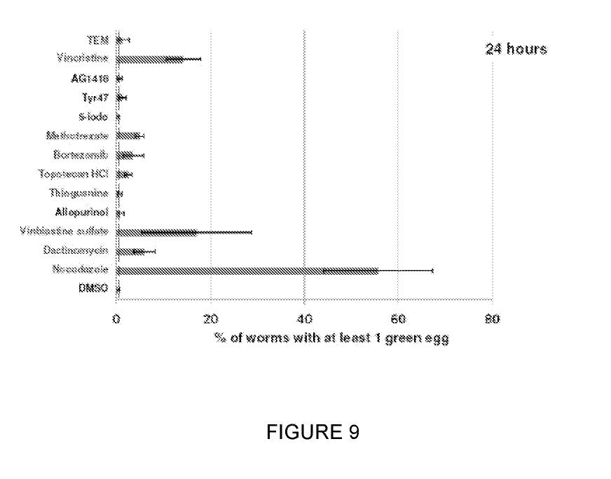

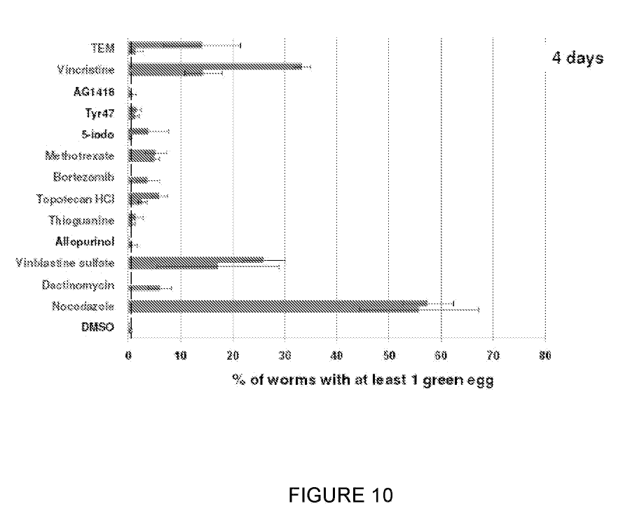

Each well contained 750 μl of M9, the drug at a final concentration of 100 μM, concentrated bacteria and young adults+eggs. The cultures were examined at 24 hours and 4 days. The results are presented in FIG. 9 (24 hours) and FIG. 10 (4 days).

These findings indicate that aneuploidies in C. elegans can be chemically induced using GFP and luciferase as a read-out, and that the assay can accurately discriminate between previously characterized aneugenic and non-aneugenic compounds.

### High-Throughput Capacity of the System

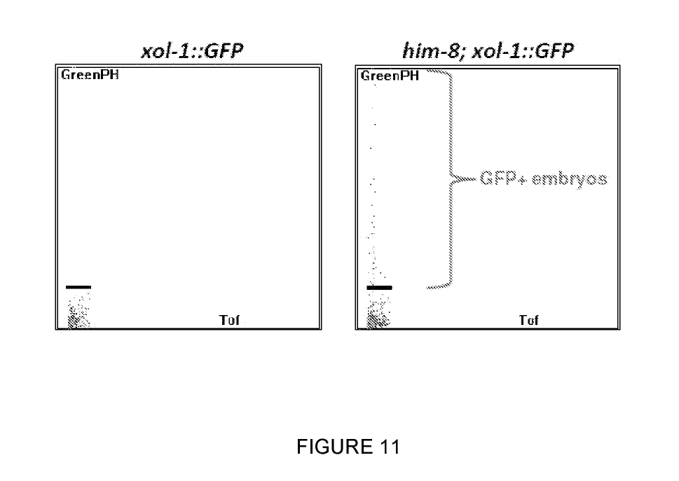

This use of the COPAS Biosort worm sorter and the Profiler analysis software in the high-throughput analysis was tested. The instruments have built-in 96-well plate capabilities (24-26) to screen two strains of worms, one that generates about 30% of males and green embryos (him-8; xol-1::GFP) by missegregation of the X chromosome during meiosis (27) and one that does not (xol-1::GFP). A large population of GFP positive eggs was easily detected in the him-8; xol-1::GFP, but not in the xol-1::GFP strain (FIG. 11) and established that, in this context, the rate of false positives (i.e. number of GFP+ eggs above threshold) was extremely low.

C. elegans has been widely used in high-throughput screens as they can be cultured in various plate sizes including 96-well plates and grown in buffer solution with food for easier dispensing and processing of the worms (15, 16, 22, 23). The sensitivity of the system will be determined in 96-well plates (that can accommodate ˜30 worms/well) by measuring the number of aneuploid embryos that can be detected per well after exposure to aneuploid agents. As luciferase assays are highly sensitive, a 1 aneuploid embryo per well detection sensitivity is expected. After treatment in 96-well plates, the worms will be exposed to the luciferase substrate and the light emitted will be measured by a luminometer equipped with a 96-well plate reader (such as the Spectromax L from Molecular Devices). Worms are not expected to require lysing, and can be directly added to the luciferin solution (20). As the strain can also be used in the context of a fluorescence screen, a high-throughput capacity for the detection of aneuploid, GFP positive embryos will also be used. For this, the COPAS Biosort worm sorter will be used and the Profiler analysis software as well, to examine and compare the strain exposed to aneugenic chemicals and to control.

### Exposure

Based on the parameters established in the previous section, the worms will be exposed to three concentrations of the chemicals present in the libraries, likely 1 nM, 1 μM and 100 μM. These concentrations span the range of concentrations which were used in the context of other C. elegans toxicological screens (15,16) and will be adjusted if necessary based on compound solubility. The length of exposure will also be determined based on initial small scale screens. Considering the length of time it takes for nuclei to progress through all the stages of meiosis and to be fertilized, two time points, 24 hours and 72 hours, are expected to allow the detection of aneuploidies generated from alteration of both early and late meiotic events. Additional time points will be added if necessary. Nocodazole will be used as a positive control and DMSO (solvent) as a negative control.

### Read-Out

As described above, the multi-assayable system permits the detection of aneuploidies in various ways. These two approaches will be combined for in-screen validation of the hits. Thus, following exposure, the worms will be washed off the plate with a physiological buffer and half will be dispensed into a 96-well plate that contains the luciferin reaction buffer for luciferase assay. All the positive wells from this first assay will be tested from the replicate plate that will be run through a high-throughput flow cytometry device (the COPAS Biosort). With this automated nematode analysis, sorting, and dispensing platform, one 96-well plate can be screened per hour. The worm sorter not only directs the sorting of GFP positive worms from GFP negative ones but when coupled to the Profiler analysis software, also allows for the localization of the fluorescence within the worm. In the screen, I will be scoring for GFP positive eggs present in the animal's mid-ventral section (uterus). Finally the sorting option of the COPAS Biosort will be used to dispense the positive worms into a new plate for direct visualization by fluorescence microscopy of the green eggs. This in-screen triple validation of the hits should provide a high accuracy. By running each compound in triplicate, the detection threshold should approximate one aneuploid embryo out of 180 embryos which is highly sensitive. As the luciferase assay component of the screen is fast (˜1 minute per plate), once the worms have been dispensed into their reading plate, the entire Phase I Toxcast 309 library at 3 concentrations in triplicate can be screened in a maximum of 1 hour per time point and the ToxCast 21 library in 11 hours of continuous screening time. Thus, the limiting factor time wise will be the number of positive hits that need to be run through the slower COPAS Biosort. The narrowing down of the potential hits by performing the luciferase assay first will be instrumental in obtaining both high accuracy and speed.

### Data Analysis

In data analysis for the initial luciferase screen, the triplicates for each compound will be distributed on three separate plates (as opposed to three wells in a row). The same will be done with the negative control (DMSO) and positive control (nocodazole). This will allow to quickly measure and analyze the raw data to extract the true hits via cross-plate normalization based on positive and negative control levels and determination of the analysis threshold. The data analysis will be carried out in close collaboration with my partners at the EPA and the NIEHS who have extensive experience in implementation of large scale screens and data mining.

Preliminary Analysis of the Defects in C. Elegans (Mitosis vs. Meiosis)

This screening strategy uses the expression of reporters as a read-out of aneuploid embryos. However, such embryos can also be generated during the early steps of mitosis independently from any meiotic defects. Although it has not been observed so far, it is also possible that the xol-1 promoter might be directly upregulated by exposure to specific compounds. In order to verify the presence of meiotic defects, a fast and reliable fixation and DAPI staining of the worms for direct visualization of the germline nuclei, their morphology and progression through the gonad will be performed. This analysis will be performed during the screen, on the same worms that were run through the COPAS Biosorter and showed the presence of green eggs. Hallmarks of impaired meiosis include enlarged nuclei, extended leptotene and zygotene stages (identifiable by the crescent shape of the nuclei), presence of DNA fragmentation and bridges and an abnormal number of homolog pairs at diakinesis. Finally, impaired meiotic progression often correlates with increased germline apoptosis which can be easily assayed by acridine orange staining of the gonad.

### Mammalian Validation

After confirming the meiotic origin of the aneuploidies observed, the novel hits from the screen can be further validated in a mammalian system. If the hits are too numerous to be all tested, they will be prioritized according to the ToxCast chemical prioritization data from the EPA. The adequate route of exposure in mice will be chosen for each chemical to establish a dose response. Six pregnant mice will be exposed per concentration of a given chemical from gestational day 11.5 to 18.5, a period encompassing the key meiotic chromosomal events. One group of treated mice will be sacrificed at 18.5 for analysis of the prophase I oocytes as described in (9) while another group will be sacrificed at post-natal day 21 and in vitro matured for analysis of the metaphase I and metaphase II oocytes as described in (28). After chromosomal spread, the frequency of aneuploidy and of other chromosomal abnormalities will be determined (28-30). These experiments will evaluate the rate of false positives from the C. elegans screen and further establish the strategy as predictive of toxicity to mammals.

### REFERENCES FOR EXAMPLE 3

1. Hunt P A, Has sold T J. Human female meiosis: what makes a good egg go bad? Trends Genet. 2008; 24(2):86-93.
2. Woo R A, Poon R Y. Gene mutations and aneuploidy: the instability that causes cancer. Cell Cycle. 2004; 3(9):1101-3.
3. Christianson R E, Sherman S L, Torfs C P. Maternal meiosis II nondisjunction in trisomy 21 is associated with maternal low socioeconomic status. Genet Med. 2004; 6(6):487-94.
4. Hunt P A. Meiosis in mammals: recombination, non-disjunction and the environment. Biochem Soc Trans. 2006; 34(Pt 4):574-7.
5. Harkonen K. Pesticides and the induction of aneuploidy in human sperm. Cytogenet Genome Res. 2005; 111(3-4):378-83.
6. Parry J M, Parry E M. The use of the in vitro micronucleus assay to detect and assess the aneugenic activity of chemicals. Mutat Res. 2006; 607(1):5-8.
7. Pacchierotti F, Ranaldi R. Mechanisms and risk of chemically induced aneuploidy in mammalian germ cells. Curr Pharm Des. 2006; 12(12):1489-504.
8. Hales B F, Barton T S, Robaire B. Impact of paternal exposure to chemotherapy on offspring in the rat. J Natl Cancer Inst Monogr. 2005 (34):28-31.
9. Susiarjo M, Hassold T J, Freeman E, Hunt P A. Bisphenol A exposure in utero disrupts early oogenesis in the mouse. PLoS Genet. 2007; 3(1):e5. PMCID: 1781485.
10. Collins F S, Gray G M, Bucher J R. Toxicology. Transforming environmental health protection. Science. 2008; 319(5865):906-7. PMCID: 2679521.
11. Kaletta T, Hengartner M O. Finding function in novel targets: C. elegans as a model organism. Nat Rev Drug Discov. 2006; 5(5):387-98.
12. Leung M C, Williams P L, Benedetto A, Au C, Helmcke K J, Aschner M, et al. Caenorhabditis elegans: an emerging model in biomedical and environmental toxicology. Toxicol Sci. 2008; 106(1):5-28. PMCID: 2563142.
13. Colaiacovo M P. The many facets of SC function during C. elegans meiosis. Chromosoma. 2006; 115(3):195-211.
14. Pawlowski W P, Cande W Z. Coordinating the events of the meiotic prophase. Trends Cell Biol. 2005; 15(12):674-81.
15. Boyd W A, McBride S J, Freedman J H. Effects of genetic mutations and chemical exposures on Caenorhabditis elegans feeding: evaluation of a novel, high-throughput screening assay. PLoS One. 2007; 2(12):e1259. PMCID: 2092392.
16. Boyd W A, Smith M V, Kissling G E, Freedman J H. Medium- and high-throughput screening of neurotoxicants using C. elegans. Neurotoxicol Teratol. 2009.
17. Kelly K O, Dernburg A F, Stanfield G M, Villeneuve A M. Caenorhabditis elegans msh-5 is required for both normal and radiation-induced meiotic crossing over but not for completion of meiosis. Genetics. 2000; 156(2):617-30. PMCID: 1461284.
18. Nicoll M, Akerib C C, Meyer B J. X-chromosome-counting mechanisms that determine nematode sex. Nature. 1997; 388(6638):200-4.
19. Hodgkin J, Horvitz H R, Brenner S. Nondisjunction Mutants of the Nematode CAENORHABDITIS ELEGANS. Genetics. 1979; 91(1):67-94. PMCID: 1213932.
20. Lagido C, McLaggan D, Flett A, Pettitt J, Glover L A. Rapid sublethal toxicity assessment using bioluminescent Caenorhabditis elegans, a novel whole-animal metabolic biosensor. Toxicol Sci. 2009; 109(1):88-95. PMCID: 2721657.
21. Watanabe M, Mitani N, Ishii N, Miki K. A mutation in a cuticle collagen causes hypersensitivity to the endocrine disrupting chemical, bisphenol A, in Caenorhabditis elegans. Mutat Res. 2005; 570(1):71-80.
22. Sieburth D, Ch'ng Q, Dybbs M, Tavazoie M, Kennedy S, Wang D, et al. Systematic analysis of genes required for synapse structure and function. Nature. 2005; 436(7050):510-7.
23. Kim J K, Gabel H W, Kamath R S, Tewari M, Pasquinelli A, Rual J F, et al. Functional genomic analysis of RNA interference in C. elegans. Science. 2005; 308(5725):1164-7.
24. Furlong E E, Profitt D, Scott M P. Automated sorting of live transgenic embryos. Nat Biotechnol. 2001; 19(2):153-6.
25. Gill M S, Olsen A, Sampayo J N, Lithgow G J. An automated high-throughput assay for survival of the nematode Caenorhabditis elegans. Free Radic Biol Med. 2003; 35(6):558-65.
26. Rea S L, Wu D, Cypser J R, Vaupel J W, Johnson T E. A stress-sensitive reporter predicts longevity in isogenic populations of Caenorhabditis elegans. Nat Genet. 2005; 37(8):894-8. PMCID: 1479894.
27. Phillips C M, Wong C, Bhalla N, Carlton P M, Weiser P, Meneely P M, et al. HIM-8 binds to the X chromosome pairing center and mediates chromosome-specific meiotic synapsis. Cell. 2005; 123(6):1051-63.
28. Allard P, Champigny M J, Skoggard S, Erkmann J A, Whitfield M L, Marzluff W F, et al. Stem-loop binding protein accumulates during oocyte maturation and is not cell-cycle-regulated in the early mouse embryo. J Cell Sci. 2002; 115(Pt 23):4577-86.
29. Allard P, Yang Q, Marzluff W F, Clarke H J. The stem-loop binding protein regulates translation of histone mRNA during mammalian oogenesis. Dev Biol. 2005; 286(1):195-206.
30. Yang Q, Allard P, Huang M, Zhang W, Clarke H J. Proteasomal activity is required to initiate and to sustain translational activation of messenger RNA encoding the stem-loop-binding protein during meiotic maturation in mice. Biol Reprod. 2010; 82(1):123-31. PMCID: 2844492.

What is claimed:

1. A method to identify a likely mammalian reproductive toxicant, comprising contacting the one or more test agents to a culture of C. elegans and analyzing the culture for meiotic disruption, wherein an increase in meiotic disruption of the C. elegans, indicates the test agent(s) is a likely mammalian reproductive toxicant.

2. The method of claim 1, wherein analyzing the culture for meiotic disruption is by assessing the culture for % male embryos in adult midventral sections, and meiotic disruption is indicated by an increase in % male embryos.

3. The method of claim 2, wherein the C. elegans contains a male specific nucleic acid reporter construct in expressible form.

4. The method of claim 3, wherein the nucleic acid reporter construct comprises a promoter from a male specific gene, operably linked to a nucleic acid encoding a reporter protein.

5. The method of claim 4, wherein analyzing comprises detecting an increase in the reporter protein as compared to an appropriate control.

6. The method of claim 4, wherein the gene is xol-1.

7. The method of claim 3, wherein the male specific nucleic acid reporter construct is xol-1-GFP.

8. The method of claim 1, wherein analyzing the culture for meiotic disruption is by an analysis selected from the group consisting of analyzing the culture for number of apoptotic nuclei, for RAD-51 foci, or levels of ATL-1, in adult germlines at zones 6 and 7 at late pachytene, and meiotic disruption is indicated by an increase in RAD-51 foci, an increase in the number of apoptotic nuclei or an increase in the level of ATL-1, analyzing the culture for chromosome morphology during diakinesis, and meiotic disruption is indicated by aberrant chromosome morphology during diakinesis, and analyzing the culture for disassembly of the synaptonemal complex and meiotic disruption is indicated by aberrant disassembly of the synaptonemal complex at from about late pachytene to about the end of diakinesis.

9. The method of claim 1, wherein the C. elegans of the culture has a mutation that affects the integrity of its cuticle.

10. The method of claim 9, wherein the cuticle mutation is selected from the group consisting of bis-1(nx3), dpy-2(e8), dpy-7(e88) and dpy-10(e128).

11. A method for assessing one or more test agents for the ability to disrupt meiosis in C. elegans, comprising:
a) providing one or more test agents, and a culture of C. elegans,
b) contacting the one or more test agents to the C. elegans within the culture,
c) determining the percentage of male embryos in adult mid-ventral sections;
wherein an increase in the percentage of male embryos, compared to an appropriate control, indicates the agent disrupts meiosis in C. elegans.

12. The method of claim 11, wherein the C. elegans of the culture has a mutation that affects the integrity of its cuticle.

13. The method of claim 12, wherein the cuticle mutation is selected from the group consisting of bis-1(nx3), dpy-2(e8), dpy-7(e88) and dpy-10(e128).

14. The method of claim 11, wherein the C. elegans contains a male specific nucleic acid reporter construct in expressible form.

15. The method of claim 14, wherein the male specific nucleic acid reporter construct is xol-1-GFP.

16. The method of claim 14, wherein the nucleic acid reporter construct comprises a promoter from a male specific gene, operably linked to a nucleic acid encoding a reporter protein.

17. The method of claim 16, wherein analyzing comprises detecting an increase in the reporter protein as compared to an appropriate control.

18. The method of claim 16, wherein the gene is xol-1.

19. The method of claim 16, wherein the reporter is selected from the group consisting of GFP and luciferase.

20. A method for assessing the biological safety of one or more test agents, comprising, contacting the one or more test agents to a culture of C. elegans and analyzing the culture for meiotic disruption, wherein an increase in meiotic disruption of the C. elegans, indicates that the test agent(s) has reduced biological safety to mammals.
